(19) United States
(12) Patent Application Publication
(10) Pub. No.: US 2026/0248547 A1
(43) Pub. Date: Aug. 27, 2026

(54) INFUSION SYSTEM WITH BALLOON ABLATION AND METHODS OF USING SAME

(71) Applicant: PAVmed, Inc., New York, NY (US)

(72) Inventors: Michael Boutillette, Norwell, MA (US); Lishan Aklog, Purchase, NY (US); Brian J. deGuzman, Paradise Valey, AZ (US); Gustavo Arnal, Milton, MA (US); Jim Pelletier, Northampton, MA (US); Liem Vu, Needham, MA (US); Mack Krell, Jamaica Plain, MA (US); Minh Duong, Worcester, MA (US)

(21) Appl. No.: 19/106,435

(22) PCT Filed: Sep. 15, 2022
(86) PCT No.: PCT/US2022/076463

§ 371 Date: Feb. 25, 2025

Related U.S. Application Data
(60) Provisional application No. 63/244,538, filed on Sep. 15, 2021.

(51) Int. Cl. A61B 18/04; A61B 18/00

(52) U.S. Cl. CPC A61B 18/04; A61B 2018/0022; A61B 2018/00577; A61B 2018/00714; A61B 2018/00791; A61B 2018/046

(57) ABSTRACT

A system for regulating fluid flow may include an elongated body, an expandable member, and an infusion device. The expandable member may be in fluid communication with the inflow and outflow lumens at a first end of the body. The infusion device may be coupled to a second end of the body and may include a first chamber configured for directing fluid from within the first chamber into the expandable member and a second chamber configured for regulating fluid flow from the expandable member into the second chamber via the outflow lumen. The infusion device may further include a pre-inflation chamber configured for determining a location and/or fitment of the expandable member prior to initiating a medical procedure.

### CROSS-REFERENCE TO RELATED APPLICATIONS

This application claims priority to, and the benefit of, U.S. Provisional Application No. 63/244,538, filed Sep. 15, 2021, for all subject matter common to both applications. The disclosure of said provisional application is hereby incorporated by reference in its entirety.

### TECHNICAL FIELD

This disclosure generally relates to infusion systems and methods for using an expandable member for ablating tissue.

### BACKGROUND

Balloon catheters are used for a wide variety of medical applications including angioplasty, stent deployment, embolectomy, and balloon occlusion of blood vessels. A standard balloon catheter has a catheter with at least one lumen and a compliant, semi-compliant or non-compliant balloon positioned coaxially around and bonded to the catheter at or near its distal tip. At least one of the catheter lumens, for example an inflation lumen, is in fluid communication with the inside of the balloon. The balloon is deployed by attaching a syringe or other infusion device to the proximal end of the catheter, so that it is in fluid communication with the catheter's inflation lumen, and injecting a volume of fluid (liquid or gas) through the inflation lumen into the balloon, inflating it to a specified volume or pressure. The balloon is deflated by withdrawing the fluid from the balloon through at least one of the catheter lumens, for example a deflation lumen, back into the reservoir of the syringe or other infusion device. The catheter may have additional lumens such as a guidewire lumen to facilitate maneuvering of the catheter within the body, infusion lumens to infuse fluid out the distal tip of the catheter into the patient and monitoring lumens to monitor pressure, temperature, or other parameters.

There are applications where it is desirable for the fluid which inflates the balloon to flow continuously into and out of the balloon while maintaining the balloon inflated at the desired volume and/or pressure. One such application would be thermal ablation balloon catheters which ablate tissue using hyper or hypothermia. Balloon catheters are useful in these applications because they can be designed to conform to the tissue to be ablated once positioned in the appropriate location. Another such application would be a drug delivery balloon catheter where the balloon serves as a reservoir for a drug to be delivered through its permeable wall.

Tissue ablation is performed throughout the body. It is frequently used to destroy abnormal tissue such as malignant tumors (e.g., liver, lung) or other non-malignant tissue (e.g., endometrial, prostatic). It is also frequently used to target structurally normal tissues for a specific therapeutic effect such as cardiac tissue ablation to treat arrhythmias and more recently renal nerve ablation (“renal denervation”) to treat refractory hypertension.

Tissue ablation is most commonly performed by applying energy to the target tissue to cause irreversible cellular injury. Common energy sources for tissue ablation include radiofrequency, microwave, laser, ultrasound, and cryo. Each source has its own specific characteristics, biophysical mechanism, advantages, and disadvantages. All of these modalities, with the exception of cryo, ultimately act by increasing the tissue temperature to cytotoxic levels for a given period of time. Cellular injury is generally reversible below 46° C. Although there is some variability in thermal sensitivity among different tissues and cell types, irreversible cellular injury generally occurs after 60 minutes at 46° C. and less than 5 minutes at 50° C.

Most clinical applications of thermal ablation have involved either large volumes of tissue (e.g., tumor ablation) or at least relatively thick tissues (e.g., cardiac ablation) where complete ablation of the target tissue is necessary for a successful therapeutic effect. Even a small volume of residual viable tissue can lead to clinical failure in the form of recurrent tumor growth, metastases from residual tumor or recurrent arrhythmias from residual pathways. For the ablation to be successful, the cells farthest from the energy source must reach the target cytotoxic temperature. The larger the distance from the energy probe to the border of the target tissue the more challenging the ablation, the more energy needs to be delivered and the higher the temperature near the probe needs to be. For example, RF ablation depends on electrical conductivity to generate heat but creating too much heat near the probe can generate charring which increases impedance and decreases the effective range of the ablation. A wide variety of technologies and techniques have been developed to accommodate the challenges of ablating across large distances using RF (e.g., multi-electrode probes, cooling, irrigation, and complex power algorithms). As a result, these tissue ablation modalities typically require a complex, external console to assure the precise amount of energy is delivered to the tissue to achieve the desired therapeutic effect. Simpler devices which use a “shotgun” approach may be ineffective or downright harmful.

The major limitation of standard balloon catheters in hyperthermic ablation applications is that the surrounding tissue serves as a powerful thermal sink. The temperature in the balloon may equilibrate with the surrounding tissue within a short period of time, shorter than the time necessary to perform the ablation, typically several minutes. For hypothermic (cryo) ablation the fluid temperature can be made so cold using liquid gases (e.g., argon, nitrogen) that the time required for the temperature to equilibrate is longer than the time it takes to ablate the tissue. For hyperthermic ablation, however, the options are more limited since the boiling temperature of most biocompatible fluids are only modestly above the temperature necessary to successfully ablate most tissues. Most tissue ablation is therefore performed using a fixed probe which is inserted into the tissue and attached to an external energy source (e.g., radiofrequency, microwave). The source continuously provides energy to the tissue as the heat dissipates into the surrounding tissue.

### SUMMARY

Non-limiting embodiments of a system for regulating fluid flow may include an elongated body, an expandable member, and an infusion device. In some embodiments, the elongated body may include an inflow lumen and an outflow lumen extending between a first end and a second end of the body. In certain embodiments, the expandable member may be in fluid communication with the inflow lumen and the outflow lumen at the first end of the body. In some embodiments, the infusion device may be coupled to the second end of the body. In certain embodiments, the infusion device may include a first chamber configured to direct fluid, in the presence of a first pressure, from within the first chamber into the expandable member via the inflow lumen, and a second chamber configured to regulate fluid flow, in the presence of a second pressure, from the expandable member into the second chamber via the outflow lumen.

In a non-limiting embodiment, a system for regulating fluid flow is disclosed herein. The system includes an elongated body having an inflow lumen and an outflow lumen extending between a first end and a second end of the body; an expandable member in fluid communication with the inflow lumen and the outflow lumen at the first end of the body; and an infusion device coupled to the second end of the body, the infusion device having (i) a first chamber configured to direct fluid flow, at a first pressure, from within the first chamber into the expandable member via the inflow lumen; and (ii) a second chamber configured to regulate the fluid flow by applying a second pressure on the fluid flow through the outflow lumen.

In some embodiments, the expandable member can inflate when fluid flow into the expandable member is greater than fluid flow out of the expandable member; and the expandable member can deflate when fluid flow into the expandable member is less than fluid flow out of the expandable member. The system can additionally include a pressurization source in fluid communication with the first chamber, the pressurization source can be configured to supply pressurized fluid to pressurize the first chamber to the first pressure. The system can additionally include a piston slidably disposed within the second chamber, where the piston can divide the second chamber into a first section and a second section, the first section being in fluid communication with the outflow lumen, displacement of the piston towards the first section decreases a volume of the first section and increases the second pressure; and displacement of the piston towards the second section increases a volume of the first section and decreases the second pressure. The system can additionally include a prime mover operatively coupled to the piston for displacing the piston within the second chamber.

In some embodiments, fluid flow out of the expandable member can increase when the second pressure decreases; and fluid flow out of the expandable member can decrease when the second pressure increases. The system can further include a back pressure chamber having a first section and a second section, the second section can be configured for being pressurized and de-pressurized; operatively coupled first and second pistons, the first piston can be slidably disposed within the first section and the second piston can be slidably disposed within the second section; and a biasing element operatively coupled to the first and second pistons for displacing the first and second pistons to increase or decrease a pressure in the first section of the back pressure chamber.

In some embodiments, the system can further include one or more orifices disposed in the outflow lumen for regulating fluid flow into the second chamber. The second chamber can further include a first section and a second section. The second section can be configured for being pressurized and de-pressurized; operatively coupled first and second pistons, the first piston can be slidably disposed within the first section and the second piston can be slidably disposed within the second section; and a prime mover can be operatively coupled to the first and second pistons for displacing the first and second pistons to increase or decrease the second pressure in the first section of the second chamber.

In some embodiments the second chamber can further include a first section and a second section. The second section can be configured for being pressurized and de-pressurized; operatively coupled first and second pistons, the first piston can be slidably disposed within the first section and the second piston can be slidably disposed within the second section; and a biasing element can be operatively coupled to the first and second pistons for displacing the first and second pistons to increase or decrease the second pressure in the first section of the second chamber.

In some embodiments, the system can further include one or more orifices disposed in the outflow lumen for regulating fluid flow into the second chamber. The second chamber can further include a first enclosure and a second enclosure, and operatively coupled first and second pistons. The first piston can be slidably disposed within the first enclosure and can divide the first enclosure into a first section and a second section. The second piston can be slidably disposed within the second enclosure and can divide the second enclosure into a third section and a fourth section. In some embodiments, pressurizing the first section and de-pressurizing the third section can increase the second pressure within the first section of the second chamber; and de-pressurizing the first section and pressurizing the third section can decrease the second pressure within the first section of the second chamber.

In some embodiments, the system can further include a piston slidably disposed between the first and second chambers; and a prime mover operatively coupled to the piston for displacing the piston to increase or decrease the first and second pressures. The system can additionally include a storage enclosure having a storage chamber in fluid communication with the first chamber. The storage enclosure can be configured for receiving expanded fluid from the first chamber into the storage chamber and transferring fluid from the storage chamber to the first chamber. The system can further include a piston slidably disposed within the storage enclosure and a biasing element operatively coupled to the piston for increasing or decreasing a volume of the fluid within the storage chamber.

In some embodiments, the system can further include a third chamber configured for pressurization and de-pressurization; a fourth chamber configured for pressurization and de-pressurization; a first piston slidably disposed between the first and second chambers; and a second piston slidably disposed between the third and fourth chambers. The first and second pistons can be operatively coupled such that displacing the first and second pistons increases or decreases the first and second pressures. The system can further include a pressurization source in fluid communication with the third and fourth chambers. The system can be configured for concurrently pressurizing one of the third and fourth chambers and de-pressurizing the other of the third and fourth chambers. In some embodiments, pressurizing the third chamber and de-pressurizing the fourth chamber can decreases the first pressure and can increases the second pressure; and de-pressurizing the third chamber and pressurizing the fourth chamber can increases the first pressure and can decreases the second pressure.

In some embodiments, the system can further include at least one controller; at least one heating element disposed within the fluid in the first chamber; and at least one temperature sensor disposed within the fluid in the first chamber. The at least one heating element and the at least one temperature sensor can be operatively coupled to the at least one controller configured for maintaining a temperature of the fluid in the first chamber within a predefined range.

In some embodiments, the system can further include a pre-inflation chamber configured to direct fluid, in the presence of a third pressure, into and out of the expandable member via a pre-inflation lumen in fluid communication with the expandable member via the outflow lumen.

In some embodiments, the system can further include a first piston which can be slidably disposed within the first chamber, the first piston dividing the first chamber into a first section and a second section, the first section being in fluid communication with the inflow lumen; a second piston can be slidably disposed within the second chamber, the second piston dividing the second chamber into a first section and a second section, the first section being in fluid communication with the outflow lumen; and a third piston slidably disposed within the pre-inflation chamber, the third piston dividing the pre-inflation chamber into a first section and a second section, the first section being in fluid communication with the pre-inflation lumen. Displacement of the first piston towards the first section of the first chamber can decrease a volume of the first section of the first chamber and thereby increase the first pressure; and displacement of the piston towards the second section of the first chamber can increase a volume of the first section of the first chamber and thereby decrease the first pressure. Displacement of the second piston towards the first section of the second chamber can decrease a volume of the first section of the second chamber and thereby increase the second pressure; and displacement of the second piston towards the second section of the second chamber can increase a volume of the first section of the second chamber and thereby decrease the second pressure. Displacement of the third piston towards the first section of the pre-inflation chamber can decrease a volume of the first section of the pre-inflation chamber and thereby increase the third pressure; and displacement of the third piston towards the second section of the pre-inflation chamber can increase a volume of the first section of the pre-inflation chamber and thereby decrease the third pressure. In some embodiments the system can further include one or more valves configured to inhibit fluid flow into and out of the first and second chambers such that, prior to initiating a procedure, the expandable member can be at least partially inflated to determine a location and/or fitment of the expandable member by engaging the valves to inhibit fluid flow into and out of the first and second chambers and at least partially inflating the expandable member by increasing the third pressure to direct fluid from the pre-inflation chamber into the expandable member. In some embodiments, after determining the location and/or fitment of the expandable member, the third pressure can be configured to be decreased to extract fluid from the expandable member into the pre-inflation chamber and thereby deflate the expandable member. In some embodiments, during a procedure, the expandable member can be inflated or deflated by regulating the flow of fluid through the expandable member and by inhibiting the flow of fluid into the pre-inflation chamber. The expandable member can be configured to inflate when fluid flow into the expandable member is greater than fluid flow out of the expandable member. Fluid from the expandable member can be configured to be extracted into the first and/or second chambers and thereby deflate the expandable member. The system can further include a transfer lumen extending between the first and second chambers configured for transferring fluid from the second chamber to the first chamber after deflating the expandable member at the end of the medical procedure. The expandable member can be configured to deflate when fluid flow into the expandable member is less than fluid flow out of the expandable member.

In some embodiments the system can further include at least one controller; at least one heating element disposed within the fluid in the first chamber; and at least one temperature sensor disposed within the fluid in the first chamber. The at least one heating element and the at least one temperature sensor can be operatively coupled to the at least one controller configured for maintaining a temperature of the fluid in the first chamber within a predefined range.

In some embodiments the system can further include a first pressure sensor configured to measure a first pressure within the inflow lumen and a second pressure sensor configured to measure a second pressure within the outflow lumen; and a controller to which the first pressure sensor and second pressure sensor are operatively coupled. The controller can be configured to operate the infusion device to control at least one of the first pressure and the second pressure within the first chamber and the second chamber, respectively, based on the measured first pressure within the inflow lumen and the measured second pressure within the outflow lumen.

In a non-limiting embodiment, a method for regulating fluid flow is disclosed. The method includes providing an elongated body having an inflow lumen and an outflow lumen extending between a first end and a second end of the body; providing an expandable member in fluid communication with the inflow lumen and the outflow lumen at the first end of the body; providing an infusion device coupled to the second end of the body, the infusion device having: a first chamber in fluid communication with the inflow lumen; and a second chamber in fluid communication with the outflow lumen; inserting the expandable member into a tube; and advancing the expandable member to a target site within the tube.

In some embodiments, the method can further include the steps of (1) applying a first pressure with the first chamber whereby fluid within the first chamber is directed into the expandable member via the inflow lumen; and/or (2) applying a second pressure with the second chamber to regulate fluid flow from the expandable member to the second chamber via the outflow lumen.

In some embodiments, the method can further include the step of regulating a pressure in the expandable member by regulating the second pressure. The method can further include the step of providing one or more orifices configured for regulating fluid flow into the second chamber. The method can further include the step of providing one or more orifices configured for regulating a pressure in the expandable member. The method can further include the step of providing a transfer lumen configured for inhibiting fluid flow from the first chamber to the second chamber. The method can further include the step of transferring fluid from the second chamber to the first chamber via the transfer lumen by inhibiting fluid flow in the inflow and outflow lumens; and making the second pressure greater than the first pressure. The method can further include the step of transferring fluid from the expandable member to the second chamber via the outflow lumen by inhibiting fluid flow in the inflow lumen; and decreasing the second pressure. The method can further include the steps of transferring fluid from the expandable member to the first chamber via the inflow lumen; and/or transferring fluid from the second chamber to the first chamber via the transfer lumen. The method can further include the steps of providing a piston separating the first and second chambers; and operating the piston to concurrently change the first and second pressures.

In some embodiments, the method can further include the steps of increasing the first pressure and decreasing the second pressure by moving the piston towards a distal end of the second chamber; and/or decreasing the first pressure and increasing the second pressure by moving the piston towards a proximal end of the second chamber. The method can further include the step of transferring fluid from the second chamber to the first chamber via the transfer lumen by inhibiting fluid flow in the inflow and outflow lumens; and making the second pressure greater than the first pressure. The method can further include the step of transferring fluid from the expandable member to the second chamber via the outflow lumen by inhibiting fluid flow in the inflow lumen; and decreasing the second pressure. The method can further include the steps of transferring fluid from the expandable member to the first chamber via the inflow lumen; and/or transferring fluid from the second chamber to the first chamber via the transfer lumen.

In some embodiments, the method can further include the steps of providing a temperature controller; providing at least one heating element disposed within the first chamber and operatively coupled to the temperature controller; providing at least one temperature sensor disposed within the first chamber and operatively coupled to the temperature controller; and maintaining a temperature of the fluid in the first chamber within a predefined range by operating the at least one heating element responsive to a sensed temperature measured by the at least one temperature sensor.

In some embodiments, the method can further include the step of providing a pre-inflation chamber in fluid communication with the expandable member via a pre-inflation lumen in fluid communication with the outflow lumen. In some embodiments, prior to initiating a medical procedure, the method can further include the step of determining a location and/or fitment of the expandable member by inhibiting fluid flow into and out of the first and second chambers; and directing fluid from the pre-inflation chamber into the expandable member to at least partially inflate the expandable member. After determining the location and/or fitment of the expandable member, the method can further include the step of deflating the expandable member by extracting fluid from the expandable member into the pre-inflation chamber. During a medical procedure, the expandable member can be inflated or deflated by regulating the flow of fluid through the expandable member and inhibiting the flow of fluid into the pre-inflation chamber. The method can further include the step of inflating the expandable member by adjusting the first and/or second pressures such that an amount of fluid flowing into the expandable member is greater than the amount of fluid flowing out of the expandable member.

In some embodiments, the method can further include the step of deflating the expandable member by extracting the fluid from the expandable member into the first and/or the second chambers. After deflating the expandable member at the end of the medical procedure, the method further includes the step of transferring fluid from the second chamber to the first chamber. The method can further include the step of deflating the expandable member by adjusting the first and/or second pressures such that an amount of fluid flowing into the expandable member is less than the amount of fluid flowing out of the expandable member.

In some embodiments, the method can further include the steps of providing a temperature controller; providing at least one heating element disposed within the first chamber and operatively coupled to the temperature controller; providing at least one temperature sensor disposed within the first chamber and operatively coupled to the temperature controller; and/or maintaining a temperature of the fluid in the first chamber within a predefined range by operating the at least one heating element responsive to a sensed temperature measured by the at least one temperature sensor. The method can further include the steps of measuring a first pressure within the inflow lumen and a second pressure within the outflow lumen, and/or operating the infusion device to control at least one of the first pressure and the second pressure within the first chamber and the second chamber, respectively, based on the measured first pressure within the inflow lumen and the measured second pressure within the outflow lumen.

In a non-limiting embodiment, a system for regulating flow and temperature of a fluid is disclosed. The system includes an elongated body having an inflow lumen and an outflow lumen extending between a first end and a second end of the body; an inflatable member in fluid communication with the inflow lumen and the outflow lumen at the first end of the body; and an infusion device coupled to the second end of the body, the infusion device having (i) a chamber configured to direct fluid flow, at a pressure, from within the chamber into the inflatable member via the inflow lumen; (ii) a heating chamber configured to regulate temperature of fluid disposed therein; and (iii) a transfer lumen for directing fluid from the heating chamber to the chamber.

In some embodiments, the system can further include a three-way shut-off valve disposed in-line with the transfer lumen and the outflow lumen. The three-way shut-off valve can be configured to allow (i) fluid to flow from the heating chamber to the chamber; and/or (ii) fluid to flow from the chamber to the inflatable member. The system can further include a piston disposed within the chamber, the piston can be configured to draw a fluid from the heating chamber and to apply the pressure to direct the fluid from the chamber to the inflatable member. In some embodiments, the piston can be configured to apply a pressure to the chamber to advance the fluid through the inflow lumen, through the inflatable member, to the heating chamber. The chamber can be configured to pre-inflate the inflatable member with a second fluid.

### BRIEF DESCRIPTION OF THE DRAWINGS

FIGS. 1A and 1B illustrate a non-limiting exemplary embodiment of a generic system for regulating fluid flow in an expandable member;

FIG. 2 illustrates a non-limiting exemplary embodiment of an infusion system for regulating fluid flow in an expandable member;

FIG. 3 illustrates a non-limiting exemplary embodiment of another infusion system for regulating fluid flow in an expandable member;

FIG. 4 illustrates a non-limiting exemplary embodiment of yet another infusion system for regulating fluid flow in an expandable member;

FIG. 5 illustrates a non-limiting exemplary embodiment of another infusion system for regulating fluid flow in an expandable member;

FIG. 6 illustrates a non-limiting exemplary embodiment of yet another infusion system for regulating fluid flow in an expandable member;

FIG. 7 illustrates a non-limiting exemplary embodiment of another infusion system for regulating fluid flow in an expandable member;

FIG. 8 illustrates a non-limiting exemplary embodiment of yet another infusion system for regulating fluid flow in an expandable member;

FIG. 9 illustrates a non-limiting exemplary embodiment of another infusion system for regulating fluid flow in an expandable member;

FIG. 10 illustrates a non-limiting exemplary embodiment of yet another infusion system for regulating fluid flow in an expandable member;

FIG. 11 illustrates a non-limiting exemplary embodiment of another infusion system for regulating fluid flow in an expandable member;

FIG. 12 illustrates a non-limiting exemplary embodiment of yet another infusion system for regulating fluid flow in an expandable member;

FIG. 13 illustrates a non-limiting exemplary embodiment of another infusion system for regulating fluid flow in an expandable member;

FIG. 14 illustrates a non-limiting exemplary embodiment of yet another infusion system for regulating fluid flow in an expandable member;

FIGS. 15A and 15B illustrate a non-limiting exemplary embodiment of another infusion system for regulating fluid flow in a non-expandable member;

FIGS. 16A, 16B, 16C, and 16D illustrate non-limiting exemplary embodiments of infusion systems; and

FIGS. 17A, 17B, 17C, and 17D illustrate a non-limiting exemplary embodiment of operation of valves of the embodiment of FIG. 15B.

### DETAILED DESCRIPTION

There are applications where it is desirable for the fluid which inflates an expandable member, e.g., a balloon, to flow continuously into and out of the member while maintaining the member inflated at the desired volume and/or pressure to assure continuous tissue contact. One such application can be thermal ablation systems which can ablate tissue using hyper or hypothermia. In some such applications the surrounding tissue may serve as a heat sink which rapidly dissipates the thermal energy from the balloon, i.e., equilibrating. A possible solution to eliminate or minimize equilibrating with surrounding tissue may be to circulate a hot or cold fluid into and out of an expandable member while maintaining the member at an inflation which may be critical to assure tissue contact and thermal transfer into the target tissue. Maintaining such an equilibrium may require continuous flow of fluid with precise matching of flow into and out of the expandable member.

In various non-limiting exemplary embodiments, systems utilizing expandable members may include one or more features configured to restrict the flow of fluid into and/or out of the expandable member (one such feature being referred to herein as a “flow restriction”). In one aspect, restricting the flow out of the expandable member could enable the expandable member to inflate naturally as fluid is initially introduced. In another aspect, once fully inflated, the flow restriction could restrict the rate at which fluid can be pushed out of the expandable member and thereby help keep the expandable member at a constant volume and pressure with less inflow.

Flow restrictions may be implemented in numerous ways in the present systems. In an embodiment, the dimensions of the outflow conduit may be varied to provide a flow restriction. Generally speaking, the smaller the inner diameter of the outflow conduit and the longer the length of the outflow conduit, the lesser the fluid flow rate through the outflow lumen and thus the greater the flow restriction. Accordingly, in various embodiments, the outflow conduit may be configured with a smaller inner diameter and/or a longer length than the inflow conduit to provide a flow restriction. The magnitude of the flow restriction (i.e., the flow rate achievable through the outflow conduit relative to the flow rate achievable through the inflow conduit) may be controlled through selection of these parameters. In another embodiment, the outflow port may be dimensioned to restrict the flow of fluid out of the expandable member and into the outflow conduit. For example, the diameter of the outflow port could be made smaller than that of the outflow conduit. The outflow port could be designed with such dimensions, or a structure (e.g., a cover) or mechanism (e.g., a valve) may be positioned at or in the outflow port to produce a similar effect. In yet another embodiment, a flow restriction could be achieved using a valve with a certain cracking pressure. This may facilitate inflation of the expandable member by preventing (as opposed to restricting) fluid from escaping the expandable member until a certain pressure inside the expandable member is reached-namely, one corresponding with full inflation of the expandable member and keyed to a desired inflow rate. In still another embodiment, a static head pressure could be applied to the system that must be overcome. One of ordinary skill in the art will recognize other flow restrictions suitable for restricting the flow of fluid into and/or out of the expandable member. For example, in some embodiments the expandable member may be non-compliant, or non-expandable, such that the introduction of fluid into the member does not expand the member. In such an embodiment, the flow restriction can be controlled via other variables as there is less, if no, concern that the member will change shape due to the introduction of fluid pressure.

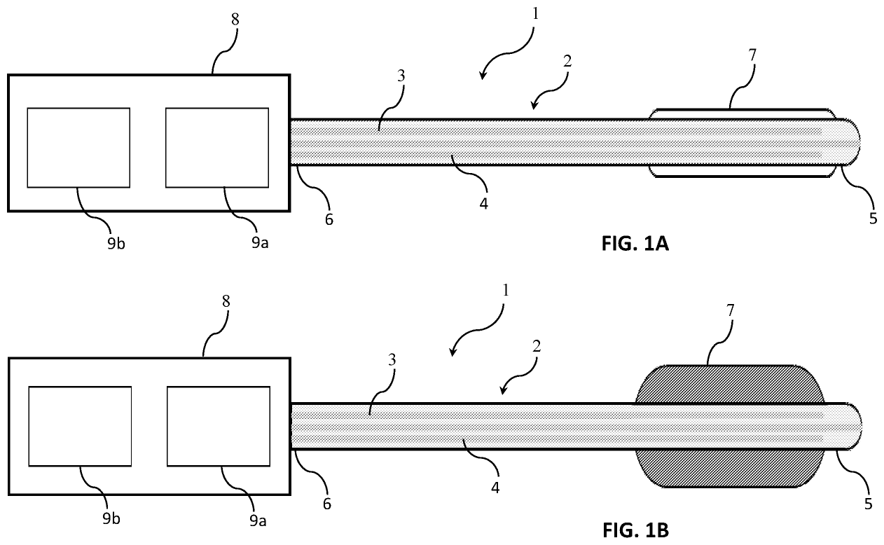

FIGS. 1A and 1B illustrate a non-limiting exemplary embodiment of a system 1 for regulating fluid flow in a system used for tissue ablation. In some embodiments, the system 1 may include an elongated body 2 having one or more inflow lumens 3 and one or more outflow lumens 4 extending between a first end 5 and a second end 6 of the body 2. In certain embodiments, the system 1 may include an expandable member 7 in fluid communication with the inflow and outflow lumens 3 and 4, respectively, proximate the first end 5 of the body 2. The expandable member 7 is depicted in a collapsed or deflated state in FIG. 1A and in an expanded or inflated state in FIG. 1B.

In a non-limiting exemplary embodiment, the inflow and outflow lumens 3 and 4, respectively, within the body 2 could be arranged for minimizing thermal transfer therebetween, and between the lumens and a patient's blood and tissues. In some embodiments, the body 2 may include thermal insulating material or air pockets for thermally insulating the lumens 3 and 4 from each other.

In a non-limiting exemplary embodiment, the expandable member 7 may be constructed of any material conducive to thermal transfer and can be compliant, semi-compliant or non-compliant in nature. Examples of materials for use in connection with expandable member 7 can include, without limitation, polyurethane, nylon, polyethylene, PET, PEBAX, or a combination thereof, or any material capable of transferring heat including metal. In some embodiments, the elongated body 2 may be made of similar materials. In certain embodiments, the one or more inflow and outflow lumens 3 and 4, respectively, may be in fluid communication with the expandable member 7 through one or more orifices (not shown). In operation, the one or more inflow lumens 3 could be used for supplying fluid to the expandable member 7 and the one or more outflow lumens 4 could be used for extracting fluid from the expandable member 7. In a non-limiting exemplary embodiment, the system 1 may be configured such that the flow of fluid in the lumens and through the expandable member 7 can be reversed. In some embodiments, when the flow is reversed, the lumen 3 could become the outflow lumen and the lumen 4 could become the inflow lumen. In certain embodiments, the elongated body 2 may include one or more additional lumens, as necessary, for guidewires, infusion, monitoring, and other functionalities.

In a non-limiting exemplary embodiment, the system 1 may include an infusion device 8 coupled to the second end 6 of the body 2. In some embodiments, the infusion device 8 can supply a fluid to the expandable member 7 via the inflow lumen 3, and fluid from the expandable member 7 can be returned to the infusion device 8 via the outflow lumen 4. In certain embodiments, the flow of fluid into and out of the expandable member 7 may be regulated to maintain the fluid therewithin at a desired volume and/or pressure. In some embodiments, the infusion device 8 may heat or cool the fluid to a pre-determined temperature. In some embodiments, the fluid may be replenished, replaced, recirculated, or recycled.

Various exemplary embodiments of infusion devices such as, for example, infusion device 7 are described in further detail herein with reference to the accompanying figures wherein like numerals designate like components. In general, non-limiting exemplary embodiments of infusion devices may include one or more fluid storage chambers such as, for example, first and second chambers 9a and 9b in fluid communication with the expandable member 7 via inflow and outflow lumens 3 and 4, and one or more pressurization sources, e.g., pumps, bellows, pistons, etc., (not shown) coupled to each of the one or more fluid storage chambers 9a and 9b. In some embodiments, the expandable member 7 and the first chamber 9a may be in fluid communication via the inflow lumen 3, and the expandable member 7 and the second chamber 9b may be in fluid communication via the outflow lumen 4. In some embodiments, the one or more pressurization sources may be operable for regulating the flow of fluid into and out of the lumens 3 and 4 and through the expandable member 7. As such, manipulating the pressures in the one or more fluid storage chambers 9a and 9b could affect the volume of fluid retained within the expandable member 7. In certain embodiments, the pressure in the one or more fluid storage chambers in fluid communication with the expandable member 7 via the outflow lumen 4 could operate as a “back pressure” on the fluid exiting the expandable member 7 via the outflow lumen 4 to throttle the flow rate of fluid exiting the expandable member 7. Accordingly, increasing the back pressure, e.g., the pressure within second chamber 9b, may decrease the volume of fluid exiting the expandable member 7, and decreasing the back pressure, e.g., the pressure within the second chamber 9b, may increase the volume of fluid exiting the expandable member 7.

In some embodiments, the pressure in the one or more fluid storage chambers in fluid communication with the expandable member 7 via the inflow lumen 3 might affect the volume of fluid flowing into the expandable member 7. For instance, increasing the pressure, e.g., the pressure within the first chamber 9a, could increase the volume of fluid entering the expandable member 7, and decreasing the pressure, e.g., the pressure within the first chamber 9b, could decrease the volume of fluid entering the expandable member 7.

In view thereof, the volume of fluid retained within or flowing through the expandable member 7 can be regulated by manipulating the pressures in the one or more chambers 9a and 9b, i.e., by regulating the volume of fluid flowing through the expandable member 7 via the inflow and outflow lumens 3 and 4. For instance, the volume of fluid retained within the expandable member 7 can be increased, i.e., the volume of fluid flow through the expandable member 7 can be increased, by increasing the volume of fluid entering the expandable member 7 via the inflow lumen 3. The volume of fluid entering via the inflow lumen 3 can be increased by increasing the pressure in the associated fluid storage chambers while concurrently decreasing, or limiting, the volume of fluid exiting the expandable member 7 via the outflow lumen 4. The decrease in volume of fluid exiting the expandable member 7 can be accomplished by increasing the pressure, e.g., the back pressure, in the associated fluid storage chambers. Accordingly, the expandable member 7 can be expanded or inflated and partially or fully collapsed or deflated by regulating the volume of the fluid flowing therethrough and/or retained therein. An increase in the volume of fluid retained within the expandable member 7 can further expand or inflate the expandable member 7 whereas a decrease in the volume of fluid retained within the expandable member 7 can partially or fully collapse or deflate the expandable member. For instance, increasing the volume of the fluid retained within the expandable member 7 might expand or inflate the expandable member. As will be apparent to one of ordinary skill, restricting the expandable member 7 from expanding or inflating while the volume of the retained fluid increases could increase the pressure applied by the expandable member 7 onto the surrounding tissue. Alternatively, instead of varying the pressure of fluid through the expandable member 7, the flow rate of fluid through the expandable member 7 can be varied. For example, when the flow rate of fluid into and out of the expandable member is maintained at equal, but opposite, rates then the volume of the expandable member 7 can be in a stasis, or maintained. Additionally, the increase, or decrease, of pressure within the expandable member 7 can change the area of the expandable member 7 that is in contact with the surrounding tissue.

In a non-limiting exemplary embodiment, the one or more pressures within the one or more chambers 9a and 9b can be adjusted for controlling the volume of the fluid flowing into and out of the expandable member 7. Accordingly, the one or more pressures within the one or more chambers 9a and 9b can be adjusted for expanding or inflating the expandable member 7. For instance, during treatment, the pressure can be adjusted to ensure a net inflow of the fluid into the expandable member 7, i.e., by setting the one or more pressures such that the volume of the fluid entering the expandable member 7, is greater than the volume of fluid exiting the expandable member 7. Likewise, the one or more pressures within the one or more chambers 9a and 9b may be adjusted for partially or fully collapsing or deflating the expandable member 7. For example, during and/or after treatment, ensuring a net outflow of the fluid from the expandable member 7, i.e., by setting the one or more pressures such that the volume of the fluid exiting the expandable member 7, is greater than the volume of fluid entering the expandable member 7. Deflation of the expandable member 7 can also be accomplished by ensuring no fluid flows into the expandable member 7 and extracting the fluid within the expandable member 7, for instance at the end of treatment.

In the following detailed description of the numerous non-limiting exemplary embodiments of systems for inflating and deflating an expandable member by regulating the flow of fluid therethrough, it should be clearly understood that the term “lumen” and “lumens” are used inter-changeably. Accordingly, the singular term “lumen” might represent “one or more lumens” and the term “one or more lumens” might represent a single lumen. Additionally, unless explicitly states otherwise, the term “expandable member” refers to the embodiments of the expandable member 7 as described with reference to FIGS. 1A and 1B. Furthermore, unless explicitly stated otherwise, the term “infusion device” refers to the embodiments of the infusion device 8 as described with reference to FIGS. 1A and 1B.

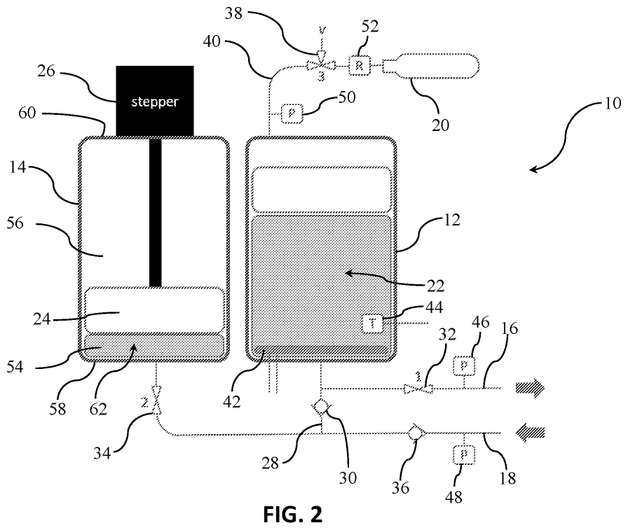

FIG. 2 illustrates a non-limiting embodiment of an infusion device 10 generically illustrated as infusion device 8 in FIGS. 1A and 1B, for regulating fluid flow in an expandable member such as, for example, expandable member 7 of infusion system 1 in FIGS. 1A and 1B. In a non-limiting exemplary embodiment, infusion device 10 can include a first chamber 12 and a second chamber 14. The infusion device 10 may be coupled to a second end (e.g., second end 6 in FIGS. 1A and 1B) of an elongated body (e.g., elongated body 2 in FIGS. 1A and 1B) having inflow and outflow lumens 16 and 18 (e.g., inflow and outflow lumens 3 and 4 in FIGS. 1A and 1B). The infusion device 10 can include a pressurization source 20 coupled to the first chamber 12. In some embodiments, pressurizing the first chamber 12, for instance using the pressurization source 20, might direct the fluid 22 from the first chamber 12 into the expandable member (not shown) via the inflow lumen 16. The fluid may flow through the expandable member and into the second chamber 14 via the outflow lumen 18. In a non-limiting embodiment, the second chamber 14 may be pressurized to regulate the volume of fluid flowing from the expandable member into the second chamber 14. In a non-limiting exemplary embodiment, a piston or plunger 24 operatively coupled to a prime mover 26, e.g., a stepper motor, may be slidably disposed within the second chamber 14 for regulating the pressure therewithin. It should be appreciated that the piston 24, by design, may divide the second chamber 14 into first and second sections 54 and 56 and may be operable to change the volume of the first section 54 to accommodate incoming fluid from the expandable member via the outflow lumen 18.

To that end, operating the prime mover 26, e.g., a stepper motor, to displace the piston 24 towards a first end 58 of the second chamber 14 could increase the back pressure, specifically the pressure within the first section 54, and decrease the volume of fluid entering the first section 54 of the second chamber 14 from the expandable member via the outflow lumen 18. Further, operating the prime mover 26 to displace the piston 24 towards a second end 60 of the second chamber 14 could decrease the back pressure, specifically the pressure within the first section 54, and increase the volume of fluid entering the first section 54 of the second chamber 14 from the expandable member via the outflow lumen 18. Accordingly, regulating the pressures within the first chamber 12 and within the first section 54 of the second chamber 14 may affect the volume of fluid retained within the expandable member by modulating the volume of fluid entering and exiting the expandable member. As such, the expandable member can be expanded or inflated by increasing the volume of fluid retained within the expandable member, and the expandable member can be fully or partially collapsed or deflated by decreasing the volume of fluid retained within the expandable member. In some embodiments, the first chamber 12 and second chamber 14 can be actuated with the same pressure source, e.g., both pressurization source 20 or both actuated with a prime mover 26.

In a non-limiting embodiment, the infusion device 10 can generally include a transfer lumen 28 having a one-way valve 30, a shut-off valve 32 in the inflow lumen 16, a shut-off valve 34 and a one-way valve 36 in the outflow lumen 18, and a valve 38 in the lumen 40 extending between the first chamber 12 and the pressurization source 20.

The expandable member, for example expandable member 7 as shown in FIGS. 1A and 1B, can be expanded and/or collapsed by appropriately adjusting the pressures within the first chamber 12 and first section 54 of the second chamber 14. For expanding or inflating the expandable member, for example during treatment, the valve 38 may be opened to establish fluid communication between the pressurization source 20 and the first chamber 12 for directing pressurized fluid from the pressurization source 20 into the first chamber 12 via the lumen 40. The shut-off valve 32 may be opened to establish fluid communication between the first chamber 12 and the expandable member whereby fluid 22 may be directed from the pressurized first chamber 12 into the expandable member via the inflow lumen 16. The one-way valve 30 in the transfer lumen 28 may be configured for inhibiting direct flow of fluid from the first chamber 12 into the second chamber 14 (i.e., the first section 54) via the transfer lumen 28 and may ensure that the fluid 22 from the pressurized first chamber 12 flows into only the inflow lumen 16. The shut-off valve 34 may be operated to establish fluid communication between the expandable member and the second chamber 14 (i.e., the first section 54), whereby fluid from the expandable member may be directed into the second chamber 14 via the outflow lumen 18. The volume of the fluid flowing from the expandable member into the second chamber 14 via the outflow lumen 18 may be regulated, i.e., increased or decreased, by displacing the piston 24 towards the first end 58 or towards the second end 60 of the second chamber 14 to regulate the pressure, e.g., the back pressure, within the second chamber 14.

For collapsing or deflating the expandable member, for instance during or after completion of treatment, pressurization of the first chamber 12 may be stopped by closing the valve 38 to inhibit fluid flow in the lumen 40, closing the shut-off valve 32 to inhibit fluid flow in the inflow lumen 16, opening, or maintaining, the shut-off valve 34 to establish or maintain fluid communication between the expandable member and the first section 54 of the second chamber 14, and decreasing the pressure within the second chamber 14 (i.e., first section 54) by actuating the prime mover 26 to displace the piston 24 towards the second end 60, to create a vacuum pressure in the outflow lumen 18. The one-way valve 30 may be configured for inhibiting fluid flow from the first chamber 12 into the second chamber 14 (i.e., the first section 54). The one-way valve 36 may be configured for inhibiting fluid flow into the expandable member via the outflow lumen 18. Accordingly, the expandable member could be partially or fully deflated by extracting the fluid from the expandable member into the second chamber 14 via the outflow lumen 18.

In a non-limiting embodiment, the fluid 62 in the second chamber 14 from the expandable member may be returned to the first chamber 12, for example, to reset the system for the next procedure. In some embodiments, the pressure within the second chamber 14 may be increased by operating the prime mover 26 to displace the piston 24 towards the first end 58, closing the shut-off valve 32 to inhibit fluid flow in the inflow lumen 16, opening the shut-off valve 34 to establish or maintain fluid communication in the outflow lumen 18, and decreasing the pressure within the first chamber 12. In certain embodiments, the valve 38 may be opened to vent the first chamber 12 to the atmosphere or to a vacuum source to inhibit the first chamber 12 from pressurizing.

In a non-limiting embodiment, the infusion device 10 may include one or more energy sources 42 and may also include one or more temperature sensors 44 disposed within the fluid 22 in the first chamber 12 and operatively coupled to a controller (not shown). In some embodiments, the controller may operate the one or more energy sources 42, e.g., a thermal heat source, to heat and/or cool the fluid 22 within the first chamber 12 in response to the sensed temperatures from the one or more temperature sensors 44. The one or more temperature sensors 44 can be in communication with the energy source to allow for a feedback loop to ensure that the fluid 22 is maintained at a predetermined temperature by the user. In certain embodiments, for example, a controller (not shown) may determine an average sensed temperature of the fluid 22 within the first chamber 12 and operate the one or more energy sources 42 to regulate the average temperature of the fluid 22 at a pre-defined value or within a pre-defined range.

In some embodiments, the infusion device 10 may include one or more pressure sensors or transducers for monitoring the pressures at one or more locations. In some embodiments, the infusion device 10 may include a pressure sensor 46 for monitoring the pressure of the fluid within the inflow lumen 16, a pressure sensor 48 for monitoring the pressure of the fluid within the outflow lumen 18, and a pressure sensor 50 for monitoring the pressure of the fluid within the lumen 40. The illustrated locations of the pressure sensors 46, 48 and 50 are exemplary and should not be considered as limiting. For instance, while the pressure sensor 46 is illustrated “downstream” of the shut-off valve 32, in some embodiments the pressure sensor 46 may be located “upstream” of the shut-off valve 32. Also, the number of pressure sensors should not be considered as being limited to three. In certain embodiments, the infusion device 10 may include more than three or less than three pressure sensors.

In a non-limiting embodiment, the one or more pressure sensors may be operatively coupled to a controller (not shown). In some embodiments, the controller may be the same as the controller for heating and/or cooling the fluid 22 within the first chamber 12. In certain embodiments, the one or more pressure sensors may be coupled to a separate controller. In some embodiments, the one or more pressure sensors may be used for monitoring and/or displaying the pressures. In certain embodiments, the one or more pressure sensors may be used for monitoring and/or displaying the pressures and for operating the infusion device 10 by controlling the operational status of one or more components such as, for instance, one or more valves, one or more prime movers, etc.

In a non-limiting embodiment, the infusion device 10 may include a regulator 52 disposed in the lumen 40 for regulating the pressurization fluid from the pressurization source 20 into the first chamber 12.

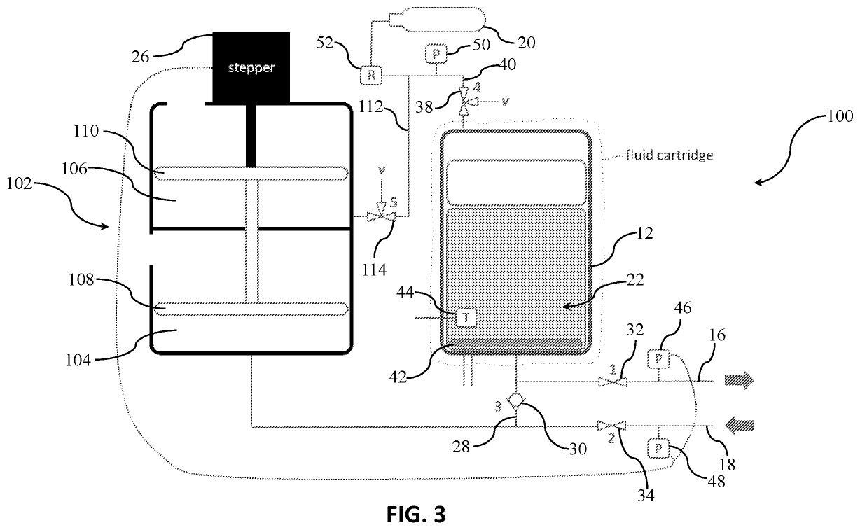

FIG. 3 illustrates a non-limiting embodiment of another infusion device 100 such as, for example, infusion device 8 for regulating fluid flow in an expandable member such as, for example, expandable member 7. In a non-limiting embodiment, infusion device 100 may include several components that are substantially the same as or similar to the components of other exemplary infusion devices described herein. All such same or similar components are designated by like numerals. In the interest of brevity, the following description of infusion device 100 focuses primarily on those components that are substantially different and/or operate or function differently from those of other exemplary infusion devices described herein.

In a non-limiting exemplary embodiment, the infusion device 100 may include a second chamber 102 configured for adjusting the back pressure for regulating the flow of fluid from the expandable member via the outflow lumen 18. In some embodiments, the second chamber 102 can include a first section 104 in fluid communication with the outflow lumen 18, a second section 106, and a pressurization system having a prime mover, e.g., a stepper motor, 26 operatively coupled to a first piston 108 and a second piston 110. In certain embodiments, the first piston 108 may be slidably disposed within the first section 104 and the second piston 110 may be slidably disposed within the second section 106. In some embodiments, the prime mover 26, the first piston 108 and second piston 110 may be operatively coupled such that the first and second pistons 108 and 110 can be concurrently displaced within their respective sections 104 and 106 by operating the prime mover 26. In a non-limiting exemplary embodiment, operating the prime mover 26 in a first direction could pressurize the first and second sections 104 and 106, and operating the prime mover 26 in a second direction opposite the first direction could de-pressurize the first and second sections 104 and 106. Pressurizing the first section 104 could increase the back pressure and de-pressurizing the first section could decrease the back pressure.

In a non-limiting embodiment, the expandable member may be inflated and deflated by adjusting the pressures within the first chamber 12 and in the first section 104 of the second chamber 102 as described herein above. For example, pressurizing the first chamber 12 could direct the fluid 22 into the expandable member via the inflow lumen 16 and adjusting the back pressure, i.e., changing the pressure within the first section 104, could regulate the flow of the fluid from the expandable member into the first section 104 via the outflow lumen 18. Pressurizing the first section 104 while operating the shut-off valves 32 and 34 to inhibit fluid flow in their respective lumens 16 and 18, and de-pressurizing the first chamber 12 could divert fluid from within the first section 104 into the first chamber 12 via the transfer lumen 28 and the one-way valve 30.

In a non-limiting embodiment, the second section 106 may be in fluid communication with the pressurization source 20 via a lumen 112 having a valve 114. In some embodiments, when operating the prime mover 26 to pressurize the first section 104, the second section 106 may be vented, and not pressurized, by opening the valve 114 to the atmosphere and/or a vacuum source. In certain embodiments, the first section 104 may be de-pressurized, i.e., the back pressure may be decreased by pressurizing the second section 106, such that the first piston 108 may be displaced out of the first section 104. In some embodiments, the second section 106 may be pressurized by operating the valve 114 to direct pressurized fluid from the pressurization source 20 into the second section 106 via the lumen 112 and operating the valve 38 to inhibit flow into the first chamber 12 via the lumen 40. In certain embodiments, the prime mover 26 may be operative when pressurizing the second section 106 with the fluid from the pressurization source 20. Alternatively, the prime mover 26 can be left in a neutral state such that the pressurization can freely

In a non-limiting embodiment, the second section 106 may not be vented when, or while, the first section 104 is pressurized. In some embodiments, the valve 114 may be operated to permit pressurized fluid to flow into the lumen 112 from the second section 106. For example, the pressurized fluid from the second section 106 may be directed into the pressurization source 20 such as, e.g., a tank. In certain embodiments, the pressurized fluid from the second section 106 may be directed into the first chamber 12. In some embodiments, the pressurized fluid from the second section 106 may be directed into both the first chamber 12 and the pressurization source 20.

In a non-limiting embodiment, the infusion device 100 may include one or more energy sources 42, one or more temperature sensors 44, one or more pressure sensors, e.g., 46, 48 and 50, one or more regulators 52, and one or more controllers for regulating the temperature of the fluid 22 within the first chamber 12 and for operating the infusion system. In some embodiments, the prime mover 26, the one or more pressure sensors, e.g., sensors 46 and 48, the one or more regulators 52, the one or more energy sources 42, and the one or more temperature sensors 44 may be operatively coupled to the same controller. In certain embodiments, the prime mover 26, the one or more pressure sensors, e.g., sensors 46 and 48, and the one or more regulators 52 may be operatively coupled to a first controller, and the one or more energy sources 42 and the one or more temperature sensors 44 may be operatively coupled to a second controller.

In a non-limiting exemplary embodiment, the first chamber 12 may be a fluid cartridge or may be substantially similar to a fluid cartridge, to ensure contaminant free flushing fluid.

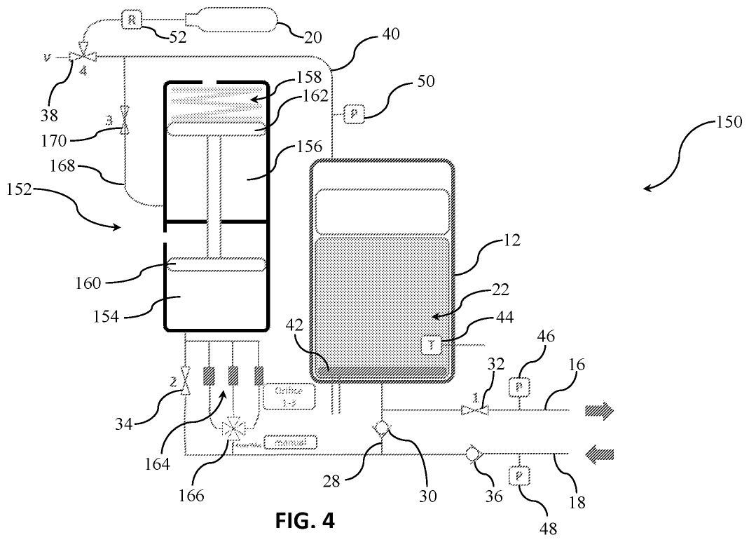

FIG. 4 illustrates a non-limiting embodiment of yet another infusion device 150 such as, for example, infusion device 8 for regulating fluid flow in an expandable member such as, for example, expandable member 7. In a non-limiting embodiment, the infusion device 150 may include several components that are substantially the same as or similar to the components of other exemplary infusion devices described herein. All such same or similar components are designated by like numerals. In the interest of brevity, the following description of infusion device 150 focuses primarily on those components that are substantially different and/or operate or function differently from those of other infusion devices described herein.

In a non-limiting embodiment, the infusion device 150 may include a second chamber 152 configured for adjusting the back pressure for regulating the flow of fluid from the expandable member via the outflow lumen 18. In some embodiments, the second chamber 152 may include a first section 154 in fluid communication with the outflow lumen 18, a second section 156, and a biasing element 158. In certain embodiments, the biasing element 158 may be operatively coupled to a first piston 160 and a second piston 162. In some embodiments, the first piston 160 may be slidably disposed within the first section 154 and the second piston 162 may be slidably disposed within the second section 156. In some embodiments, the biasing element 158, the first piston 160 and the second piston 162 may be operatively coupled, or relatively fixed together, such that the first and second pistons 160 and 162 can be displaced within their respective sections 154 and 156 by adjusting the pressure within the second section 156. In a non-limiting embodiment, pressurizing the second section 156 can displace the pistons 160 and 162, toward the biasing element 158, and out of their respective first and second section 154 and 156 whereby the first section 154 can de-pressurize and decrease the back pressure in the outflow lumen 18. When de-pressurizing the second section 156, the energy stored within the biasing element 158 can displace the first and second pistons 160 and 162 into their respective first and second sections 154 and 156 whereby the first section 154 can pressurize and increase the back pressure.

In a non-limiting embodiment, the second section 156 may be in fluid communication with the pressurization source 20 via a lumen 168 having a valve 170. In some embodiments, the second section 156 may be pressurized by operating the valve 170 to establish or maintain fluid communication in the lumen 168 whereby pressurized fluid can be diverted from the pressurization source 20 into the second section 156 to adjust the backpressure in the outflow lumen 18. In certain embodiments, the valves 38 and 170 may be operated to vent the second section 156 to the atmosphere and/or a vacuum source. In a non-limiting embodiment, the first chamber 12 may be pressurized by operating the valve 170 to inhibit fluid flow in the lumen 168 whereby pressurized fluid from the pressurization source 20 can be diverted through the valve 38 and into the first chamber 12 via the lumen 40. The valve 170 may be a variable flow valve and the pressures in the first chamber 12 and in the first and second sections 154 and 156 may be adjustable by modulating the valve 170. In certain embodiments, the valve 170 can be a manually adjusted valve, a remotely actuated valve, or an automatically actuated valve.

In a non-limiting exemplary embodiment, the expandable member may be inflated and deflated by adjusting the pressures within the first chamber 12 and in the first section 154 of the second chamber 152 as described herein above. Briefly, pressurizing the first chamber 12 can direct fluid 22 into the expandable member via the inflow lumen 16 and adjusting the back pressure, i.e., by changing the pressure within the first section 154, can regulate the flow of the fluid from the expandable member into the first section 154 via the outflow lumen 18. Pressurizing the first section 154 with the shut-off valve 32 inhibiting fluid flow in the inflow lumen 16, operating the shut-off valve 34 to enable or maintain fluid communication in the outflow lumen 18, and de-pressurizing the first chamber 12 can divert fluid 22 from within the first section 154 into the first chamber 12 via the transfer lumen 28 and the one-way valve 30. The one-way valve 36 inhibits fluid flow into the expandable member via the outflow lumen 18.

In a non-limiting embodiment, the infusion device 150 can include one or more orifices 164 and a multi-port valve 166 for regulating the amount of fluid entering the first section 154 from the expandable member via the outflow lumen 18.

The one or more orifices 164 can be configured for “metering” the flow of fluid into the first section 154, in addition to, or in place of, adjusting the backflow. In some embodiments, each of the one or more orifices 164 may be configured for permitting the same amount of fluid to flow therethrough into the first section 154, and the total amount of fluid entering the first section 154 may be regulated by opening or closing one or more ports of the multi-port valve 166. For example, opening two ports of the multi-port valve 166 can direct the fluid through two of the orifices 164 doubling the volume of fluid entering the first section 154 from the expandable member via the outflow lumen 18. In certain embodiments, each of the one or more orifices 164 may be calibrated or configured for permitting different amounts of fluid to flow therethrough into the first section 154, and the amount of fluid entering the first section can be regulated by opening or closing different ports of the multi-port valve 166. For instance, opening the “first port” can direct fluid through the “first orifice” configured for a “first flow rate”, and opening the “second port” can direct fluid through the “second orifice” configured for a “second flow rate” different from the “first flow rate”. In some embodiments, more than one port may be activated for directing the fluid through corresponding orifices each having different flow rates. Accordingly, the total amount of fluid entering the first section 154 can be regulated by opening or closing one or more ports of the multi-port valve 166.

In a non-limiting embodiment, the second section 156 may not vent when the first section 154 is pressurized. In some embodiments, the pressurized fluid from the second section 156 may be directed into the pressurization source 20 such as, for example, a tank. In certain embodiments, the pressurized fluid from the second section 156 may be directed into the first chamber 12. In some embodiments, the pressurized fluid from the second section 156 may be directed into both the first chamber 12 and the pressurization source 20.

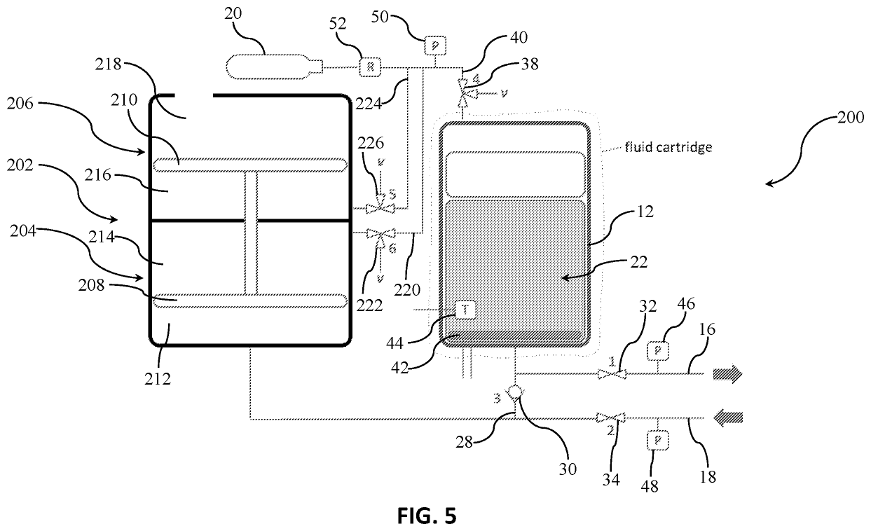

FIG. 5 illustrates a non-limiting embodiment of another infusion device 200 such as, for example, infusion device 8 for regulating fluid flow in an expandable member such as, for example, expandable member 7. In a non-limiting embodiment, the infusion device 200 may include several components that are substantially the same as or similar to the components of other exemplary infusion devices described herein. All such same or similar components are designated by like numerals. In the interest of brevity, the following description of infusion device 200 focuses primarily on those components that are substantially different and/or operate or function differently from those of other infusion devices described herein.

In a non-limiting embodiment, the infusion device 200 may include a second chamber 202 configured for adjusting the back pressure for regulating the flow of fluid from the expandable member via the outflow lumen 18. In some embodiments, the second chamber 202 may include a first enclosure 204 and a second enclosure 206. In certain embodiments, the second chamber 202 may include operatively coupled first and second dividers, e.g., pistons or plungers, 208 and 210 slidably disposed within the first and second enclosures 204 and 206, respectively. The pistons, or plungers, 208 and 210 can be fixed together such that movement of one of the plungers affects movement of the other. In some embodiments, the pistons 208, 210 can otherwise, or additionally, be dividers. The first divider 208 can divide the first enclosure 204 into a first section 212 and a second section 214. The second divider 210 can divide the second enclosure 206 into a third section 216 and a vented fourth section 218. In some embodiments, the first section 212 can be in fluid communication with the expandable member via the outflow lumen 18, and the second section 214 may be in fluid communication with the pressurization source 20 via a lumen 220 having a valve 222. In certain embodiments, the third section 216 may be in fluid communication with the pressurization source 20 via a lumen 224 having a valve 226.

In a non-limiting embodiment, the back pressure, i.e., the pressure within the first section 212 may be adjusted by displacing the divider 208 into or out of the first section 212. In some embodiments, displacing the divider 208 into the first section 212 could pressurize the first enclosure 212, i.e., increase the back pressure, and decrease the flow of fluid from the expandable member to the first section 212 via the outflow lumen 18. In certain embodiments, displacing the divider 208 out of the first section 212, toward the second enclosure 206, can de-pressurize the first section 212, i.e., decrease the back pressure, and increase the flow of fluid from the expandable member to the first section 212 via the outflow lumen 18. This functionality can be beneficial to maintain the volume of fluid within the expandable member. In some embodiments, the first section 212 can be pressurized, i.e., the back pressure increased, by pressurizing the second section 214 with pressurized fluid from the pressurization source 20 via the lumen 220 and through the valve 222. In certain embodiments, the valve 226 may be operated to the atmosphere and/or a vacuum source for venting the third section 216 while the second section 214 is being pressurized. In some embodiments, the first section 212 may be de-pressurized, i.e., the back pressure decreased, by pressurizing the third section 216 with pressurized fluid from the pressurization source 20 via the lumen 224 and through the valve 226. In certain embodiments, while the third section 216 is being pressurized, the second section 214 may be vented by opening the valve 222 to the atmosphere and/or a vacuum source.

In a non-limiting embodiment, the expandable member may be inflated and deflated by adjusting the pressures within the first chamber 12 and within the first section 212 of the first enclosure 204 of the second chamber 202. Briefly, pressurizing the first chamber 12 can direct the fluid 22 into the expandable member via the inflow lumen 16 and adjusting the back pressure, i.e., changing the pressure within the first section 212, can regulate the flow of the fluid from the expandable member into the first section 212 via the outflow lumen 18. Pressurizing the first section 212 while the shut-off valves 32 and 34 are closed to inhibit fluid flow in the inflow and outflow lumens 16 and 18 and de-pressurizing the first chamber 12 can divert fluid from within the first section 212 into the first chamber 12 via the transfer lumen 28 and the one-way valve 30, to reset the system. The one-way valve 30 may be configured for inhibiting fluid flow into the expandable member via the outflow lumen 18, while allowing fluid to flow from the first section 212 to the first chamber 12.

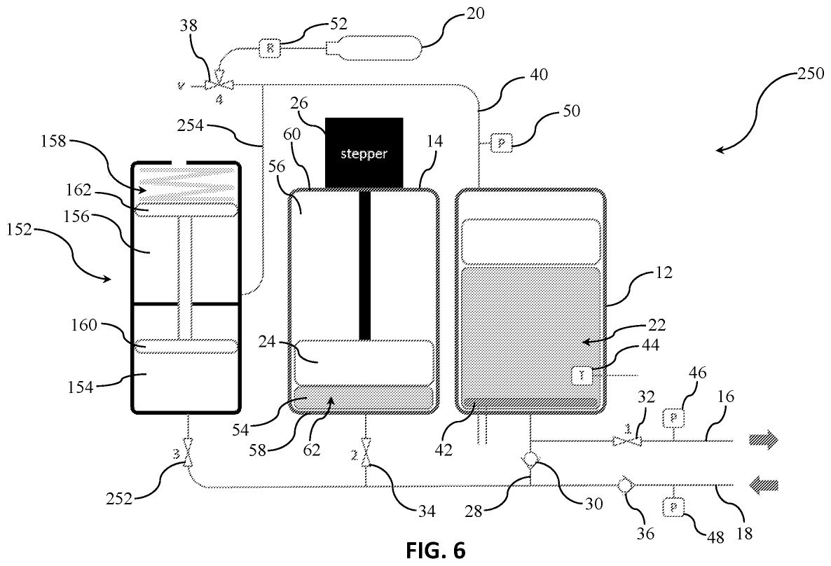

FIG. 6 illustrates a non-limiting embodiment of yet another infusion device 250 such as, for example, infusion device 8 for regulating fluid flow in an expandable member such as, for example, expandable member 7. In a non-limiting embodiment, the infusion device 250 may include several components that are substantially the same as or similar to the components of other exemplary infusion devices described herein. All such same or similar components are designated by like numerals. In the interest of brevity, the following description of infusion device 250 focuses primarily on those components that are substantially different and/or operate or function differently from those of other infusion devices described herein.

In a non-limiting embodiment, the infusion device 250 may be a hybrid of the infusion devices 10 and 150 described herein above with reference to FIGS. 2 and 4. In some embodiments, the infusion device 250 may include one first chamber 12 and two back pressure or second chambers 14 and 152. Two back pressure, or second chambers, 14 and 152 can allow for a redundant system in case of failure of one of the two chambers and/or provide added granularity to the back pressure system to allow for a higher degree of accuracy in maintaining the pressure and volume in the expandable member. In the interest of brevity, the following description focuses primarily on the operation of the infusion device 250.

In a non-limiting embodiment, the expandable member may be inflated and deflated by adjusting the pressures within the first chamber 12, within the first back pressure chamber 14, and within the first section 154 of the second back pressure chamber 152 as described herein above. Briefly, pressurizing the first chamber 12 can direct the fluid 22 into the expandable member via the inflow lumen 16 and adjusting the back pressure, i.e., changing the pressure within the first back pressure chamber 14 and within the first section 154 of the second back pressure chamber 152, can regulate the flow of fluid from the expandable member into the first back pressure chamber 14 via the outflow lumen 18. Pressurizing the first back pressure chamber 14 while operating the shut-off valves 32 and 252 to inhibit fluid flow in their respective lumens and de-pressurizing the first chamber 12 can divert fluid 62 from within the first back pressure chamber 14 into the first chamber 12 via the transfer lumen 28 and the one-way valve 30. De-pressurizing the first back pressure chamber 14 while maintaining the shut-off valve 34 open and shut-off valves 32 and 252 closed could extract the fluid from within the expandable member into the first back pressure chamber 14 via the outflow lumen 18. The one-way valve 30 inhibits fluid flow from the first chamber 12 into the first back pressure chamber 14 via the transfer lumen 28, and the one-way valve 36 inhibits fluid flow into the expandable member via the outflow lumen 18.

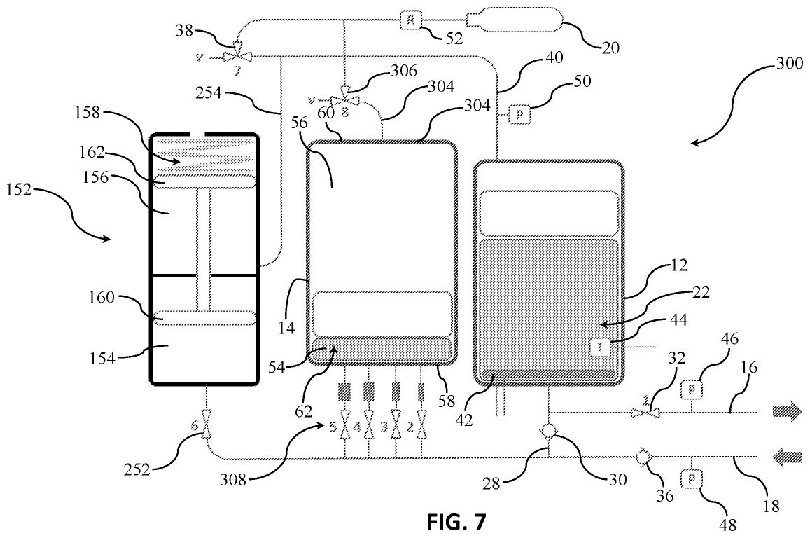

FIG. 7 illustrates a non-limiting exemplary embodiment of another infusion device 300 such as, for example, infusion device 8 for regulating fluid flow in an expandable member such as, for example, expandable member 7. In a non-limiting exemplary embodiment, the infusion device 300 may include several components that are substantially the same as or similar to the components of other exemplary infusion devices described herein. All such same or similar components are designated by like numerals. In the interest of brevity, the following description of infusion device 300 focuses primarily on those components that are substantially different and/or operate or function differently from those of other infusion devices described herein.

In a non-limiting exemplary embodiment, the infusion device 300 may be a hybrid of the infusion devices 10 and 150 described herein above with reference to FIGS. 2 and 4. In some embodiments, the infusion device 300 may include one first chamber 12 and two second or back pressure chambers 14 and 152. In certain embodiments, the first back pressure chamber 14 may be in fluid communication with the expandable member via the outflow lumen 18 and one or more orifices 308. In some embodiments, in place of the stepper 26, the first back pressure chamber 14 may be in fluid communication with the pressurization source 20 via a lumen 304 having a valve 306. In the interest of brevity, the following description focuses primarily on the operation of the infusion device 250.

In certain embodiments, the one or more orifices 308 may be configured for “metering” the flow of fluid into the first back pressure chamber 14. In some embodiments, each of the one or more orifices 308 may be configured for permitting the same amount of fluid to flow therethrough into the first back pressure chamber 14. In certain embodiments, each of the one or more orifices 308 may be calibrated or configured for permitting different amounts of fluid to flow therethrough into the first back pressure chamber 14.

In a non-limiting embodiment, the expandable member may be inflated and deflated by adjusting the pressures within the first chamber 12, within the first back pressure chamber 14, and within the first section 154 of the second back pressure chamber 152 as described herein above. Briefly, pressurizing the first chamber 12 can direct the fluid 22 into the expandable member via the inflow lumen 16 and adjusting the back pressure, i.e., changing the pressure within the first back pressure chamber 14 and within the first section 154 of the second back pressure chamber 152, can regulate the flow of fluid from the expandable member into the first back pressure chamber 14 via the outflow lumen 18. Pressurizing the first back pressure chamber 14 while operating the shut-off valves 32 and 252 to inhibit fluid flow in their respective lumens and de-pressurizing the first chamber 12 could divert fluid 62 from within the first back pressure chamber 14 into the first chamber 12 via the transfer lumen 28 and the one-way valve 30. De-pressurizing the first back pressure chamber 14 while operating the shut-off valves 32 and 252 to inhibit fluid flow in their respective lumens could extract the fluid from within the expandable member into the first back pressure chamber 14 via the outflow lumen 18 and the one or more orifices 308. The one-way valve 30 may inhibit fluid flow from the first chamber 12 into the second chamber 14 via the transfer lumen 28, and the one-way valve 36 may inhibit fluid flow into the expandable member via the outflow lumen 18.

In a non-limiting embodiment, pressurization of the first back pressure chamber 14 may be accomplished by operating the valve 306 to enable pressurized fluid from the pressurization source 20 to enter the first back pressure chamber 14. In some embodiments, the valve 306 may be operated to vent while fluid from the expandable member flows into the first back pressure chamber 14.

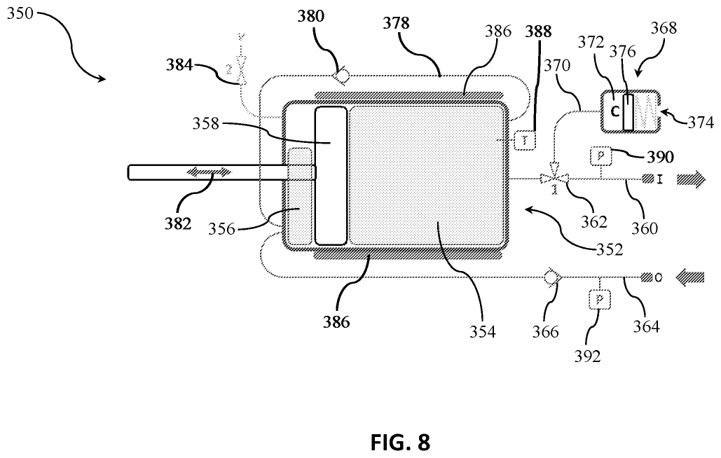

FIG. 8 illustrates a non-limiting embodiment of an alternate infusion device 350 such as, for example, infusion device 8 for regulating fluid flow in an expandable member such as, for example, expandable member 7. In a non-limiting exemplary embodiment, the infusion device 350 may include a single enclosure 352 having a first chamber 354 and a second chamber 356 separated from each other by a divider, e.g., a piston or a plunger, 358 slidably disposed within the enclosure 352.

In a non-limiting exemplary embodiment, the first chamber 354 may be in fluid communication with an expandable member (not shown) via an inflow lumen 360 having a multi-port valve 362, and the second chamber 356 may be in fluid communication with the expandable member via the outflow lumen 364 having a one-way valve 366. The one-way valve 366 may inhibit fluid flow into the expandable member via the outflow lumen 364. In certain embodiments, the infusion device 350 may include a storage enclosure 368 in fluid communication with the first chamber 354 via a lumen 370 coupled to the valve 362. In some embodiments, the storage enclosure 368 may include a storage chamber 372 and a biasing element 374 coupled to a plunger or piston 376 slidably disposed therewithin. In some embodiments, the valve 362 may be actuatable to: (i) direct fluid from the first chamber 354 into the expandable member via inflow lumen 360; or (ii) to direct fluid between the first chamber 354 and the storage chamber 372 via the lumen 370. In certain embodiments, the first and second chambers 354 and 356 may be in fluid communication via a transfer lumen 378 having a one-way valve 380. The one-way valve 380 may inhibit fluid flow from the first chamber 354 into the second chamber 356 via the transfer lumen 378, while allowing fluid flow from the second chamber 356 to the first chamber 354. In a non-limiting exemplary embodiment, the divider 358, also referred to as a piston, may be operatively coupled to a prime mover (not shown) configured for reciprocating the divider 358 in the directions indicated by the double-headed arrow 382. In some embodiments, the infusion device 350 may include a valve 384 coupled to the second chamber 356.

In a non-limiting embodiment, the infusion device 350 may be configured for inflating and deflating the expandable member by adjusting the pressures within the first and second chambers 354 and 356. For treatment, the expandable member may be inflated by pressurizing the first chamber 354, for instance by operating the prime mover to displace the divider 358 “into” the first chamber 354, to direct the fluid from within the first chamber 354 through the valve 362 into the expandable member via the inflow lumen 360. The fluid within the expandable member may be directed into the second chamber 356 via the outflow lumen 364. In some embodiments, the valve 384 may be operated to vent the second chamber 356 while displacing the plunger 358 into the first chamber 354, i.e., while pressurizing the first chamber 354. During treatment or upon completion thereof, the expandable member may be deflated by operating the valve 362 to direct fluid from the first chamber 354 into the storage chamber 372 via the lumen 370, operating the valve 384 to inhibit fluid flow therethrough, and operating the prime mover to displace the divider 358 “into” the first chamber 354 whereby the fluid from the first chamber 354 could be directed into the storage chamber 372 via the lumen 370 and the pressure within the second chamber 356 may be reduced to extract fluid from the expandable member into the second chamber 356 via the outflow lumen 364. In some embodiments, operating the prime mover to displace the divider 358 “into” the first chamber 354 can create a vacuum or a partial vacuum within the chamber 356.

In a non-limiting embodiment, upon completion of the treatment and extracting all the fluid from the expandable member, the infusion device 350 may be “reset” by operating the prime mover to displace the divider 358 “into” the second chamber 356 whereby fluid within the second chamber 356 can be directed into the first chamber 354 via the lumen 378 and fluid within the storage chamber 372 can be directed into the first chamber 354 via the lumen 370. In some embodiments, the energy stored within the biasing element 374 can assist in directing fluid from the storage chamber 372 into the first chamber 354 by displacing the plunger 376 “into” the storage chamber 372.

In a non-limiting exemplary embodiment, the infusion device 350 may include one or more energy sources 386 and one or more temperature sensors 388 disposed within the fluid in the first chamber 354 and operatively coupled to a controller (not shown). In some embodiments, the controller can operate the one or more energy sources 386 to heat and/or cool the fluid within the first chamber 354 in response to the sensed temperatures from the one or more temperature sensors 388. In certain embodiments, the controller could determine an average sensed temperature of the fluid within the first chamber 354 and operate the one or more energy sources 386 to regulate the average temperature of the fluid at a pre-defined value or within a pre-defined range.

In a non-limiting embodiment, the infusion device 350 may include one or more pressure sensors or transducers for monitoring the pressures at one or more locations to ensure that the expandable member is maintained at the desired volume as the fluid is cycled therethrough. In some embodiments, the infusion device 350 may include a pressure sensor 390 for monitoring the pressure of the fluid within the inflow lumen 360 and a pressure sensor 392 for monitoring the pressure of the fluid within the outflow lumen 364. The illustrated locations of the pressure sensors 390 and 392 are exemplary and should not be considered as limiting. For instance, while the pressure sensor 390 is illustrated “downstream” of the valve 362, in some embodiments the pressure sensor 390 could be located “upstream” of the valve 362. Also, the number of pressure sensors should not be considered as being limited to two. In certain embodiments, the infusion device 350 may include more than two or less than two pressure sensors. In some embodiments, the one or more pressure sensors may be used for monitoring and/or displaying the pressures and for operating the infusion device 350 by controlling the operational status of one or more components such as, for instance, one or more valves, one or more prime movers, etc.

In a non-limiting embodiment, the one or more prime movers, the one or more pressure sensors, the one or more energy sources, and the one or more temperature sensors may be operatively coupled to a single controller. In certain embodiments, the one or more prime movers and the one or more pressure sensors may be operatively coupled to a first controller and the one or more energy sources and the one or more temperature sensors may be operatively coupled to a second controller.

In a non-limiting exemplary embodiment, the infusion device 350 may be provided “empty” and require “charging” prior to commencing treatment. In some embodiments, sterilized fluid for charging the infusion device 350 may be provided in a bag or “cartridge” (not shown). In certain embodiments, the plunger 358 in an empty infusion device 350 may be positioned “all the way into” the first chamber 354. In some embodiments, the prime mover may be operated to displace the plunger 358 “all the way into” the first chamber 354. For filling the first chamber 354, the valve 384 may be operated to vent the second chamber 356, the fluid cartridge may be coupled to the inflow lumen 360, and the valve 362 may be operated to establish fluid communication between the first chamber 354 and the fluid cartridge. Then, the prime mover could be energized to displace the plunger 358 “out of” the first chamber 354 creating a vacuum or a partial vacuum therewithin whereby fluid from the fluid cartridge could be directed into the first chamber 354. Upon completion of the filling process, the valve 362 may be operated to inhibit fluid flow in the inflow lumen 360, the fluid cartridge may be decoupled from the inflow lumen 360, and the inflow lumen 360 may be coupled to the expandable member.

In a non-limiting exemplary embodiment, prior to commencing treatment, the one or more energy sources 386 may be energized to heat or cool the fluid within the first chamber 354. In some embodiments, wherein the fluid might expand when heated, the valve 362 may be operated to establish fluid communication between the first chamber 354 and the storage chamber 372 for storing the excess, i.e., expanded, fluid.

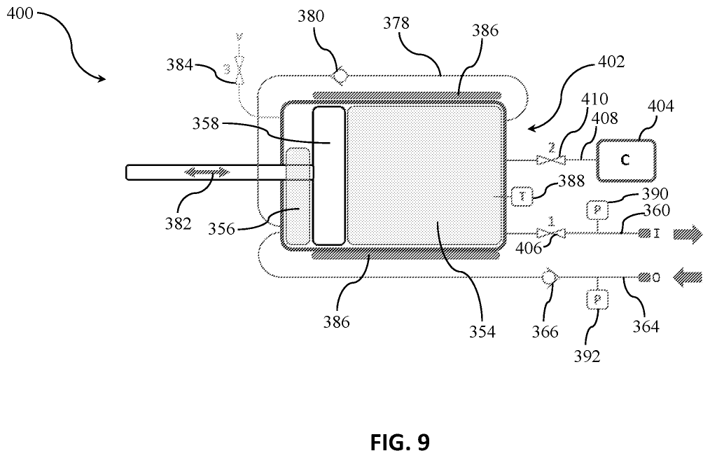

FIG. 9 illustrates a non-limiting exemplary embodiment of another infusion device 400 such as, for instance, infusion device 8 for regulating fluid flow in an expandable member such as, for example, expandable member 7. In a non-limiting exemplary embodiment, infusion device 400 may include several components that are substantially the same as or similar to the components of other exemplary infusion devices described herein. All such same or similar components are designated by like numerals. In the interest of brevity, the following description of infusion device 400 focuses primarily on those components that are substantially different and/or operate or function differently from those of other exemplary infusion devices described herein.

In a non-limiting exemplary embodiment, infusion device 400 may include an enclosure 402 having a first chamber 354, a second chamber 356, and a storage chamber 404. The first and second chambers 354 and 356 may be separated from each other by a divider, e.g., a piston or a plunger, 358 slidably disposed within the enclosure 352. In some embodiments, the first chamber 354 may be in fluid communication with an expandable member (not shown) via an inflow lumen 360 having a valve 406, and the first chamber 354 may be in fluid communication with the storage chamber 404 via a lumen 408 having a valve 410.

In a non-limiting exemplary embodiment, the infusion device 400 may be configured for inflating and deflating the expandable member by adjusting the pressures within the first and second chambers 354 and 356 in substantially the same manner as infusion device 350. Briefly, the expandable member may be inflated by pressurizing the first chamber 354 to direct the fluid from within the first chamber 354 through the valve 406 into the expandable member via the inflow lumen 360, and operating the valve 410 to inhibit fluid flow from the first chamber 354 into the storage chamber 404 via the lumen 408. The fluid within the expandable member may be directed into the second chamber 356 via the outflow lumen 364. In some embodiments, the valve 384 may be operated to vent the second chamber 356 while the first chamber 354 is being pressurized. During treatment or upon completion thereof, the expandable member may be deflated by operating the valve 406 to inhibit fluid flow from the first chamber 354 into the expandable member via the inflow lumen 360, operating the valve 410 to establish fluid communication between the first chamber 354 and the storage chamber 404 via the lumen 408, operating the valve 384 to inhibit flow, and operating the prime mover to displace the divider 358 “into” the first chamber 354 whereby the fluid from the first chamber 354 could be directed into the storage chamber 404 via the lumen 408 and the pressure within the second chamber 356 may be reduced to extract fluid from the expandable member into the second chamber 356 via the outflow lumen 364. In some embodiments, operating the prime mover to displace the divider 358 “into” the first chamber 354 could create a complete or a partial vacuum within the chamber 356.

In a non-limiting exemplary embodiment, upon completion of the treatment and extracting all the fluid from the expandable member, the infusion device 400 may be “reset” by operating the prime mover to displace the divider 358 “into” the second chamber 356 whereby fluid within the second chamber 356 may be directed into the first chamber 354 via the lumen 378 and fluid within the storage chamber 404 may be directed into the first chamber 354 via the lumen 408.

In a non-limiting exemplary embodiment, sterilized fluid for charging the infusion device 400 may be provided in a bag or “cartridge” (not shown). The plunger 358 may be positioned “all the way into” the first chamber 354. The valve 384 may be operated to vent the second chamber 356, the fluid cartridge may be coupled to the inflow lumen 360, and the valve 406 may be operated to establish fluid communication between the first chamber 354 and the fluid cartridge. Then, the prime mover may be energized to displace the plunger 358 “out of” the first chamber 354 and create a complete or partial vacuum therewithin whereby fluid from the fluid cartridge may be directed into the first chamber 354. Upon completion of the filling process, the valve 406 may be operated to “close”, the fluid cartridge may be decoupled from the inflow lumen 360, and the inflow lumen 360 may be coupled to the expandable member.

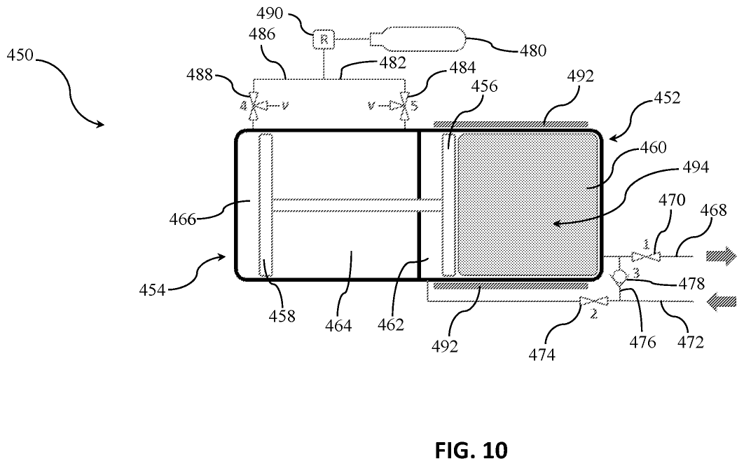

FIG. 10 illustrates a non-limiting exemplary embodiment of yet another infusion device 450 such as, for example, infusion device 8, for regulating fluid flow in an expandable member such as, for example, expandable member 7. In a non-limiting exemplary embodiment, infusion device 450 may include several components that are substantially the same as or similar to the components of other exemplary infusion devices described herein. All such same or similar components are designated by like numerals. In the interest of brevity, the following description of infusion device 450 focuses primarily on those components that are substantially different and/or operate or function differently from those of other exemplary infusion devices described herein.

In a non-limiting exemplary embodiment, infusion device 450 may include a first enclosure 452, a second enclosure 454, and operatively coupled first and second pistons or plungers 456 and 458 slidably disposed within first and second enclosures 452 and 454, respectively. In some embodiments, the first piston 456 may divide the first enclosure 452 into a first chamber 460 and a second chamber 462; and the second piston 458 may divide the second enclosure 454 into a third chamber 464 and a fourth chamber 466. In some embodiments, the first chamber 460 and the expandable member may be in fluid communication via an inflow lumen 468 having a shut-off valve 470. In certain embodiments, the second chamber 462 and the expandable member may be in fluid communication via an outflow lumen 472 having a shut-off valve 474. In some embodiments, the first and second chambers 460 and 462 may be in fluid communication via a transfer lumen 476 having a one-way valve 478 configured for inhibiting fluid flow from the first chamber 460 into the second chamber 462 via the transfer lumen 476.

In a non-limiting exemplary embodiment, the infusion device 450 may include a pressurization source 480 such as, for example, a tank or reservoir or compressor. In some embodiments, the pressurization source 480 and the third chamber 464 may be in fluid communication via a lumen 482 having a valve 484; and the pressurization source 480 and the fourth chamber 466 may be in fluid communication via a lumen 486 having a valve 488. In certain embodiments, the infusion device 450 may include one or more regulators 490 for regulating the fluid from the pressurization source 480.

In a non-limiting exemplary embodiment, the infusion device 450 may include one or more energy sources 492 for heating and/or cooling the fluid 494 within the first chamber 460. Although not shown, the infusion device 450 may include one or more temperature sensors, one or more pressure sensors, and one or more controllers. In some embodiments, the one or more energy sources 492, the one or more temperature sensors, the one or more pressure sensors, and the one or more regulators 490 may be operatively coupled to the same, single, controller. In certain embodiments, the one or more energy sources 492 and the one or more temperature sensors may be operatively coupled to a first controller, and the one or more pressure sensors and the one or more regulators 490 may be operatively coupled to a second controller. Additional and/or alternative features and/or functionalities as they relate to operating and/or monitoring the infusion device 450 have been described elsewhere as they relate to other embodiments of diffusion devices such as, for example, infusion device 8.

In a non-limiting exemplary embodiment, the infusion device 450 may be configured for inflating and/or deflating the expandable member by adjusting the pressures within the first and second chambers 460 and 462. For treatment, the expandable member may be inflated by operating the valve 484 to inhibit flow in the lumen 482 and vent the third chamber 464 to the atmosphere or a vacuum source, pressurizing the fourth chamber 466 by operating the valve 488 to establish fluid communication between the fourth chamber 466 and the pressurization source 480. Next, or simultaneously, operating the valve 470 to establish fluid communication between the first chamber 460 and the expandable member via the inflow lumen 468, and operating the valve 474 to establish fluid communication between the second chamber 462 and the expandable member via the outflow lumen 472. Pressurizing the fourth chamber 466 may displace the operatively coupled first and second pistons 456 and 458 to pressurize the first chamber 460 whereby at least a portion of the fluid 494 within the first chamber 460 can be directed into the expandable member via the inflow lumen 468, and at least a portion of the fluid within the expandable member may be directed, or drawn, into the second chamber 462 via the outflow lumen 472. In a non-limiting exemplary embodiment, the valve 474 may be configured for metering or regulating the flow of fluid therethrough, i.e., controlling the amount of fluid directed from the expandable member into the second chamber 462. For example, as a vacuum is drawn in the second chamber 462 as the piston 456 is drawn towards, and into, the first chamber 460, the valve 474 can control the draw, or vacuum, which is being applied to the outflow lumen 472 to maintain equilibrium of flow in the system.

During treatment or upon completion thereof, the expandable member may be partially or fully deflated by operating the valve 488 to inhibit flow in the lumen 486 and vent the fourth chamber 466 to the atmosphere or a vacuum source, pressurizing the third chamber 464 by operating the valve 484 to establish fluid communication between the third chamber 464 and the pressurization source 480, operating the valve 470 to establish or maintain fluid communication between the first chamber 460 and the expandable member via the inflow lumen 468, and operating the valve 474 to inhibit fluid flow to or from the second chamber 462. Pressurizing the third chamber 464 may displace the operatively coupled first and second pistons 456 and 458 whereby at least a portion of the fluid within the expandable member may be directed into the first chamber 460 via the inflow lumen 468, and at least a portion of the fluid within the expandable member may be directed into the first chamber 460 via the outflow lumen 472 and the transfer lumen 476. In a non-limiting exemplary embodiment, at least a portion of the fluid within the second chamber 462 and within the expandable member may be directed into the first chamber 466 by operating the valve 470 to inhibit fluid flow in the inflow lumen 468 and operating or retaining the valve 474 to permit fluid flow from the second chamber 462.

Upon completion of the treatment, fluid within the second chamber 462 may be directed into the first chamber 460 by de-pressurizing the first chamber 460, operating the valve 470 to inhibit fluid flow in the inflow lumen 472, and operating or retaining the valve 474 to permit fluid flow from the second chamber 462.

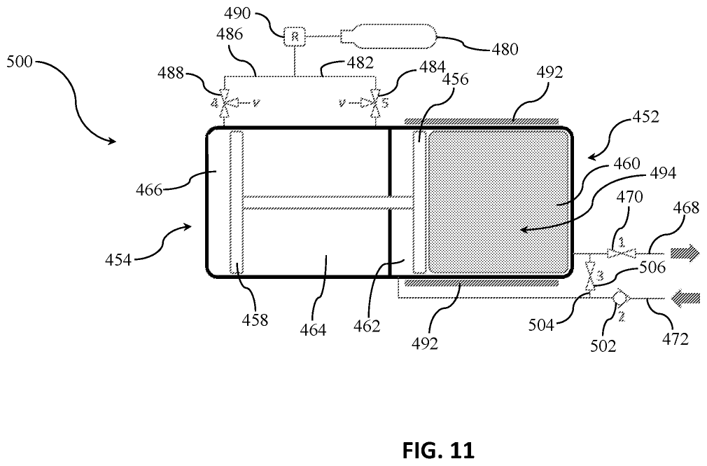

FIG. 11 illustrates a non-limiting embodiment of another infusion device 500 such as, for example, infusion device 8, for regulating fluid flow in an expandable member such as, for example, expandable member 7. In a non-limiting exemplary embodiment, infusion device 500 may include several components that are substantially the same as or similar to the components of other exemplary infusion devices described herein. All such same or similar components are designated by like numerals. In the interest of brevity, the following description of infusion device 500 focuses primarily on those components that are substantially different and/or operate or function differently from those of other exemplary infusion devices described herein.

In a non-limiting exemplary embodiment, the infusion device 500 may be substantially the same as infusion device 450 with the primary difference being in the coupling of the expandable member and the first and second chambers 460 and 462 via the inflow and outflow lumens 468 and 472. Specifically, in some embodiments, the second chamber 462 and the expandable member may be in fluid communication via the outflow lumen 472 having a one-way valve 502, and the first and second chambers 460 and 462 may be in fluid communication via a transfer lumen 504 having a shut-off valve 506. In certain embodiments, the one-way valve 502 may be configured for inhibiting fluid flow into the expandable member via the outflow lumen 472. In a non-limiting exemplary embodiment, these differences in the coupling of the expandable member and the first and second chambers 460 and 462 may affect the manner in which the expandable member is inflated and/or deflated. However, the infusion device 500 may be configured for adjusting the pressures within the first, second, third and fourth chambers 460, 462, 464 and 466 in substantially the same manner as in the infusion device 450.

In a non-limiting exemplary embodiment, for treatment with the infusion device 500, the expandable member may be inflated by pressurizing the first chamber 460, operating the valve 470 to establish fluid communication between the first chamber 460 and the expandable member via the inflow lumen 468, and operating the valve 506 to inhibit fluid flow into the first chamber 460 via the transfer lumen 504. The one-way valve 502 could be configured to permit fluid flow from the expandable member into the second chamber 462 via the outflow lumen 472 and to inhibit fluid flow into the expandable member. In a non-limiting exemplary embodiment, the transfer lumen 504 may be used for bypassing the expandable member by operating the valve 506 to permit at least a portion of the fluid 494 to flow from the first chamber 460 into the second chamber 462, or vice versa.

During treatment or upon completion thereof, the expandable member may be partially or fully deflated by de-pressurizing the first chamber 460 and operating the valve 470 to establish or retain fluid communication between the first chamber 460 and the expandable member via the inflow lumen 468. Concurrently or in the alternative, the valve 506 may be operated to establish or retain fluid communication through the transfer lumen 504 whereby fluid from the expandable member and/or fluid from within the second chamber 462 may be directed into the first chamber 460. In some embodiments, fluid from within the second chamber 462 may be directed into the first chamber 460 via the transfer lumen 504 by operating the valve 470 to inhibit fluid flow in the inflow lumen and operating the valve 506 to establish or retain fluid communication between the first and second chambers 460 and 462 via the transfer lumen 504. In certain embodiments, fluid within the expandable member may also be directed into the first chamber 460 when the infusion device 500 is operated in this configuration.

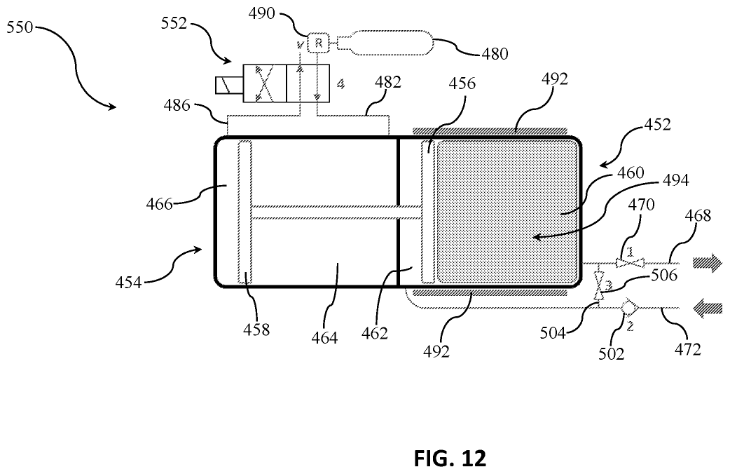

FIG. 12 illustrates a non-limiting embodiment of yet another infusion device 550 such as, for example, infusion device 8, for regulating fluid flow in an expandable member such as, for example, expandable member 7. In a non-limiting exemplary embodiment, infusion device 550 may include several components that are substantially the same as or similar to the components of other exemplary infusion devices described herein. All such same or similar components are designated by like numerals. In the interest of brevity, the following description of infusion device 550 focuses primarily on those components that are substantially different and/or operate or function differently from those of other exemplary infusion devices described herein.

In a non-limiting embodiment, the infusion device 550 is substantially the same as infusion device 500 with the primary difference being in the coupling of the pressurization source 480 with the third and fourth chambers 464 and 466. Specifically, in some embodiments, the infusion device 550 may include a multi-functional valve 552 replacing the individual valves 484 and 488 and operable for diverting or directing fluid in substantially the same manner as the replaced valves 484 and 488. In a non-limiting exemplary embodiment, the valve 552 can be operable to establish fluid communication in the lumen 486 for pressurizing the fourth chamber 466 and concurrently vent the third chamber 464. Additionally, or alternatively, the valve 552 may be operable to establish fluid communication in the lumen 482 for pressurizing the third chamber 464 and concurrently vent the fourth chamber 466. In certain embodiments, the valve 552 may be configured for inhibiting fluid flow in both lumens 482 and 486.

In a non-limiting exemplary embodiment, the infusion device 550 may be configured for inflating and/or deflating the expandable member by operating the valve 552 of the infusion device 550 in substantially the same manner as the valves 484 and 488 in the infusion device 500, and by operating the valves 470, 502 and 506 in substantially the same manner as in the infusion device 500.

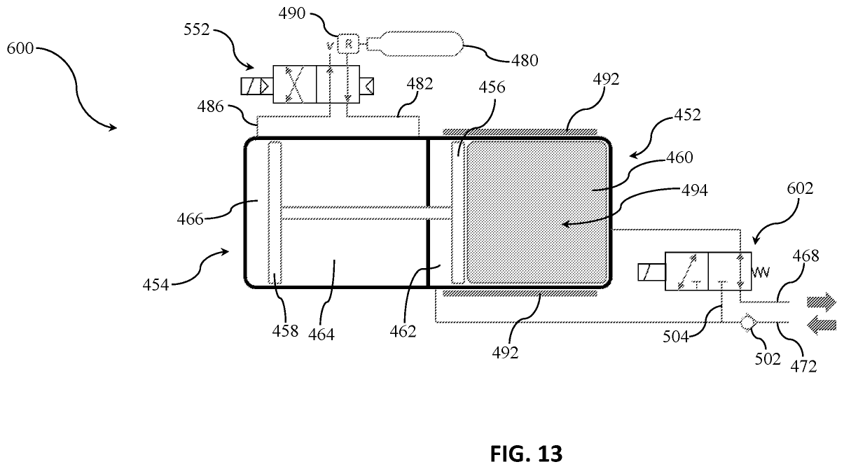

FIG. 13 illustrates a non-limiting exemplary embodiment of another infusion device 600 such as, for example, infusion device 8, for regulating fluid flow in an expandable member such as, for example, expandable member 7. In a non-limiting exemplary embodiment, infusion device 600 may include several components that are substantially the same as or similar to the components of other exemplary infusion devices described herein. All such same or similar components are designated by like numerals. In the interest of brevity, the following description of infusion device 600 focuses primarily on those components that are substantially different and/or operate or function differently from those of other exemplary infusion devices described herein.

In a non-limiting exemplary embodiment, the infusion device 600 may be substantially the same as infusion device 550 with the primary difference being in the valves for managing or directing fluid between the first and second chambers 460 and 462 and the expandable member. Specifically, in some embodiments, the infusion device 600 may include a multi-functional valve 602 replacing the valves 470 and 506 and operable for diverting or directing fluid in substantially the same manner as the replaced valves 470 and 506. In certain embodiments, the inflow, outflow, and transfer lumens 468, 472 and 504 may be coupled to or extend through the valve 602. In some embodiments, the valve 602 may be operable for diverting or directing fluid into and out of the first and second chambers 460 and 462 and into and out of the expandable member in substantially the same manner as the valves 470 and 506.

In a non-limiting embodiment, the infusion device 600 may be configured for inflating and/or deflating the expandable member by operating the valve 602 of the infusion device 600 in substantially the same manner as the valves 470 and 506 in the infusion device 550.

FIG. 14 illustrates a non-limiting embodiment of yet another infusion device 650 such as, for example, infusion device 8 for regulating fluid flow in an expandable member such as, for example, expandable member 7. In a non-limiting exemplary embodiment, the infusion device 650 may include several components that are substantially the same as or similar to the components of other exemplary infusion devices described herein. All such same or similar components are designated by like numerals. In the interest of brevity, the following description of infusion device 650 focuses primarily on those components that are substantially different and/or operate or function differently from those of other infusion devices described herein.

In a non-limiting embodiment, the infusion device 650 may be coupled to a second end (e.g., second end 6 in FIGS. 1A and 1B) of an elongated body (e.g., elongated body 2 in FIGS. 1A and 1B) having inflow and outflow lumens 16 and 18 (e.g., inflow and outflow lumens 3 and 4 in FIGS. 1A and 1B). In some embodiments, the infusion device 650 may include a first chamber 652, a back pressure or second chamber 14, and a pre-inflation chamber 654. In certain embodiments, the first chamber 652 may be in fluid communication with the expandable member via the inflow lumen 16 having the shut-off valve 32. In some embodiments, the second chamber 14 may be in fluid communication with the expandable member via the outflow lumen 18 having the shut-off valve 34. In certain embodiments, the pre-inflation chamber 654 may be in fluid communication with the expandable member via a pre-inflation lumen 656 having a shut-off valve 658 and extending between the outflow lumen 18 and the pre-inflation chamber 654. In some embodiments, the first and second chambers 652 and 14 may be in fluid communication via the transfer lumen 28 having the one-way valve 30.

In a non-limiting embodiment, the first chamber 652 may include a piston or plunger 660 slidably disposed therewithin and operatively coupled to a prime mover 662, e.g., a stepper motor A, for regulating the pressure within the first chamber 652. More specifically, the piston 660 may be operable for regulating the flow of fluid 22 into and out of the first chamber 652. It should be appreciated that the piston 660, by design, may divide the first chamber 652 into first and second sections 664a and 664b and may be operable to change the volume of the fluid 22 within the first section 664a. In certain embodiments, opening the shut-off valve 32 and operating the prime mover 662 to displace the piston 660 towards a distal end of the first chamber 652, e.g., towards the first section 664a, may direct the fluid 22 from the first section 664a into the expandable member via the inflow lumen 16. The one-way valve 30 can operate to inhibit the flow of fluid 22 from the first chamber 652 to the second chamber 14 via the transfer lumen 28 as the piston 660 is advanced towards the distal end of the first chamber 652. In some embodiments, opening the shut-off valve 32 and operating the prime mover 662 to displace the piston 660 towards a proximal end of the first chamber 652, i.e., away from the distal end of the first chamber 652 and towards the second section 664b, may extract the fluid from the expandable member into the first section 664a via the inflow lumen 16. It will be appreciated that fluid from the expandable member will not be extracted into the first chamber 652 if the shut-off valve 32 is closed while the piston 660 is displaced towards the proximal end, i.e., away from the distal end, of the first chamber 652. In certain embodiments, while the piston 660 is displaced away from the distal end of the first chamber 652, the one-way valve 30 may operate to enable (or permit) the flow of fluid 62 from the second chamber 14 to the first chamber 652 via the transfer lumen 28.

In a non-limiting embodiment, the second chamber 14 of the infusion device 650 may be configured substantially similar to, or same as, the second chamber 14 as described in reference to FIGS. 2 and 6. Accordingly, and in the interest of brevity, a detailed description of the components of the second chamber 14, as it pertains to the infusion device 650, is not repeated here. In certain embodiments, closing the shut-off valve 658, opening the shut-off valve 34, and operating the prime mover 26 to displace the piston 24 towards a distal end of the second chamber 14, e.g., towards the first section 54, could increase the back pressure, specifically the pressure within the first section 54, and decrease the rate of fluid volume entering the first section 54 of the second chamber 14 from the expandable member via the outflow lumen 18. In some embodiments, the pressure in the first chamber 652 may be maintained greater than the pressure in the second chamber 14 for ensuring the one-way valve 30 operates to inhibit the flow of fluid from the first chamber 652 to the second chamber 14 via the transfer lumen 28. In certain embodiments, closing the shut-off valve 658 may inhibit the flow of fluid from the second chamber 14 to the pre-inflation chamber 654 via the pre-inflation lumen 656 while the piston 24 is displaced towards the distal end of the second chamber 14. In some embodiments, closing the shut-off valve 658, opening the shut-off valve 34, and operating the prime mover 26 to displace the piston 24 towards a proximal end, i.e., away from a distal end, of the second chamber 14 could decrease the back pressure, specifically the pressure within the first section 54, and increase the rate of fluid volume entering the first section 54 of the second chamber 14 from the expandable member via the outflow lumen 18. In certain embodiments, the one-way valve 30 may operate to inhibit the flow of fluid 22 from the first chamber 652 to the second chamber 14 via the transfer lumen 28 irrespective of whether the pressure in the first chamber 652 is greater than or less than the pressure in the second chamber 14. In some embodiments, closing the shut-off valve 658 may inhibit the flow of fluid from the pre-inflation chamber 654 to the second chamber 14 via the pre-inflation lumen 656 while the piston 24 is displaced towards the proximal end of the second chamber 14.

In a non-limiting embodiment, the pre-inflation chamber 654 may include a piston or plunger 666 slidably disposed therewithin and operatively coupled to a prime mover 668, e.g., a stepper motor, for regulating the pressure within the pre-inflation chamber 654. More specifically, the piston 666 may be operable for regulating the flow of fluid into and out of the pre-inflation chamber 654. It should be appreciated that the piston 666, by design, may divide the pre-inflation chamber 654 into first and second sections 670a and 670b and may be operable to change the volume of the fluid within the first section 670a. In certain embodiments, closing the shut-off valves 32 and 34, opening the shut-off valve 658, and operating the prime mover 668 to displace the piston 666 towards a distal end of the pre-inflation chamber 654, e.g., towards the first section 670a, may direct the fluid from the first section 670a into the expandable member via the pre-inflation lumen 656 and the outflow lumen 18. In some embodiments, closing the shut-off valves 32 and 34, opening the shut-off valve 658, and operating the prime mover 668 to displace the piston 666 towards a proximal end of the pre-inflation chamber 654, i.e., away from the distal end of the pre-inflation chamber 654 and towards the second section 670b, may extract the fluid from the expandable member into the first section 670a via the outflow lumen 18 and the pre-inflation lumen 656. It will be appreciated that closing the shut-off valve 32 may inhibit the flow of fluid into and out of the first chamber 652 while the piston 666 is displaced towards or away from the distal end of the pre-inflation chamber 654. It will be further appreciated that closing the shut-off valve 34 may inhibit the flow of fluid into and out of the second chamber 14 while the piston 666 is displaced towards or away from the distal end of the pre-inflation chamber 654.

In a non-limiting embodiment, the infusion device 650 may be operable to pre-inflate the expandable member to test or check the location and/or fitment of the expandable member before initiating the medical procedure. In some embodiments, the expandable member may be pre-inflated by closing the shut-off valves 32 and 34, opening the shut-off valve 658, and operating the prime mover 668 to displace the piston 666 towards the distal end of the pre-inflation chamber 654. The expandable member may then be deflated by closing (or keeping closed) the shut-off valves 32 and 34, opening (or keeping open) the shut-off valve 658, and operating the prime mover 668 to displace the piston 666 towards the proximal end, i.e., away from the distal end, of the pre-inflation chamber 654.

In a non-limiting embodiment, the expandable member may be inflated during a medical procedure by opening the shut-off valves 32 and 34, closing the shut-off valve 658, and operating the prime mover 662 to displace the piston 660 towards the distal end of the first chamber 652 whereby the fluid 22 may flow, via the inflow lumen 16, from the first chamber 652 through the expandable member and into the second chamber 14 via the outflow lumen 18. The extent to which the expandable member inflates or deflates may be controlled by regulating the volumetric flow rate of the fluid therethrough. In some embodiments, the volume of fluid flowing through the expandable member and/or the flow rate may be modulated or regulated by adjusting the back pressure, i.e., the pressure in the outflow lumen 18 which may essentially, substantially, or approximately, be the same as the pressure in the second chamber 14. In certain embodiments, the pressure in the second chamber 14, i.e., the back pressure, may be regulated or adjusted by operating the prime mover 26 to displace the piston 24 towards or away from the distal end of the second chamber 14. In some embodiments, the back pressure may be increased by operating the prime mover 26 to displace the piston 24 towards the distal end of the second chamber 14. In certain embodiments, the back pressure may be decreased by operating the prime mover 26 to displace the piston 24 away the distal end of the second chamber 14. It will be appreciated that that the pressure in the first chamber 652 may be maintained substantially constant while the pressure in the second chamber 14 is adjusted for regulating the volume of fluid flowing through the expandable member.

In a non-limiting embodiment, the volume of fluid flowing through the expandable member and/or the flow rate may be modulated or regulated by adjusting the pressure in the first chamber 652 while maintaining the pressure in the second chamber 14 substantially constant. In certain embodiments, the pressure in the first chamber 652 may be regulated or adjusted by operating the prime mover 662 to displace the piston 660 towards or away from the distal end of the first chamber 652. In some embodiments, the pressure in the first chamber 652 may be increased by operating the prime mover 662 to displace the piston 660 towards the distal end of the first chamber 652. In certain embodiments, the pressure in the first chamber 652 may be decreased by operating the prime mover 662 to displace the piston 660 away the distal end of the first chamber 652.

In a non-limiting embodiment, the volume of fluid flowing through the expandable member and/or the flow rate may be modulated or regulated by concurrently adjusting the pressure in both the first chamber 652 and the second chamber 14 substantially constant.

In a non-limiting exemplary embodiment, upon completion of the medical procedure, the fluid in the second chamber 14 may be transferred to the first chamber 652 via the transfer lumen 28 by adjusting the pressure in either one or both the first chamber 652 and the second chamber 14. In some embodiments, the fluid may be transferred by increasing the pressure in the second chamber 14 while either decreasing or maintaining substantially constant the pressure in the first chamber 652. In certain embodiments, the fluid may be transferred by decreasing the pressure in the first chamber 652 while either increasing or maintaining substantially constant the pressure in the second chamber 14. The pressure in the first chamber 652 may be decreased by operating the prime mover 662 to displace the piston 660 away from the distal end of the first chamber 652; and the pressure in the second chamber 14 may be increased by operating the prime mover 26 to displace the piston 24 towards the distal end of the second chamber 14.

It should be understood that while the foregoing descriptions of the various embodiments references a single pressurization source 20, this should not be construed as a limitation. Some non-limiting exemplary embodiments of the infusion devices may include more than one pressurization sources, each pressurization source being in fluid communication with at least one chamber or section of the infusion device. For example, the exemplary embodiment of the infusion device 100 illustrated in FIG. 3 may include first and second pressurization sources 20a and 20b (not shown). In some embodiments, the first pressurization source 20a may be in fluid communication with the first chamber 12 via the lumen 40 having the valve 38; and the second pressurization source 20b may be in fluid communication with the second section 106 via the lumen 112 having the valve 114. In certain embodiments, the valves 38 and 114, respectively, may be operable to enable fluid communication between the first pressurization source 20a and the first chamber 12, and between the second pressurization source 20b and the second section 106. In some embodiments, the valve 38 may be operable to inhibit fluid communication between the first pressurization source 20a and the first chamber 12 and vent the first chamber 12 to the atmosphere or a vacuum source. In certain embodiments, the valve 114 may be operable to inhibit fluid communication between the second pressurization source 20b and the second section 106 and vent the second section 106 to the atmosphere or a vacuum source.

Likewise, the exemplary embodiment of infusion device 200 illustrated in FIG. 5 may include first, second and third pressurization sources (not shown). In some embodiments, the first pressurization source may be in fluid communication with the first chamber 12 via the lumen 40 having the valve 38; the second pressurization source may be in fluid communication with the second section 214 via the lumen 220 having the valve 222; and the third pressurization source may be in fluid communication with the third section 216 via the lumen 224 having the valve 226. In certain embodiments, the valve 38 may be operable to enable fluid communication between the first pressurization source and the first chamber 12; the valve 222 may be operable to enable fluid communication between the second pressurization source and the second section 106; and the valve 226 may be operable to enable fluid communication between the third pressurization source and the third section 216. In some embodiments, the valve 38 may be operable to inhibit fluid communication between the first pressurization source and the first chamber 12 and vent the first chamber 12 to the atmosphere or a vacuum source. In certain embodiments, the valve 222 may be operable to inhibit fluid communication between the second pressurization source and the second section 106 and vent the second section 106 to the atmosphere or a vacuum source. In some embodiments, the valve 226 may be operable to inhibit fluid communication between the third pressurization source and the third section 216 and vent the third section 216 to the atmosphere or a vacuum source.

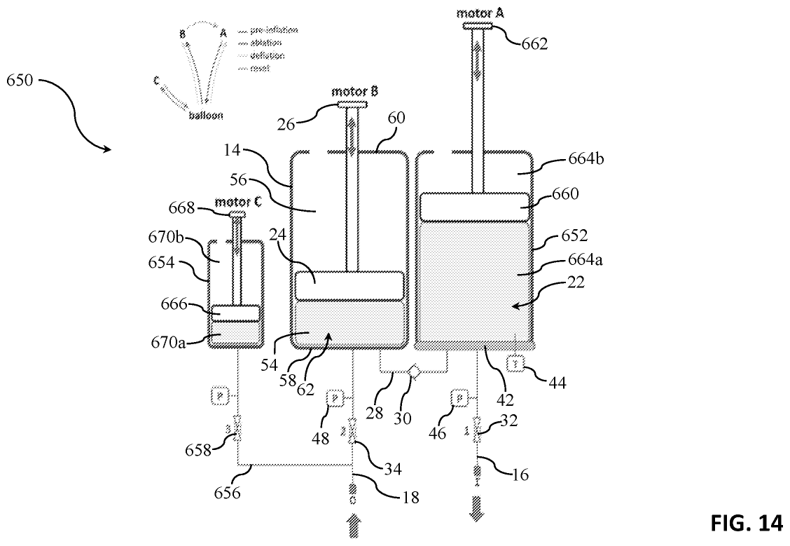

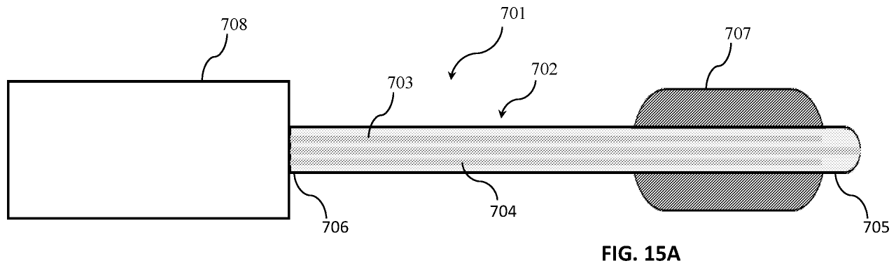

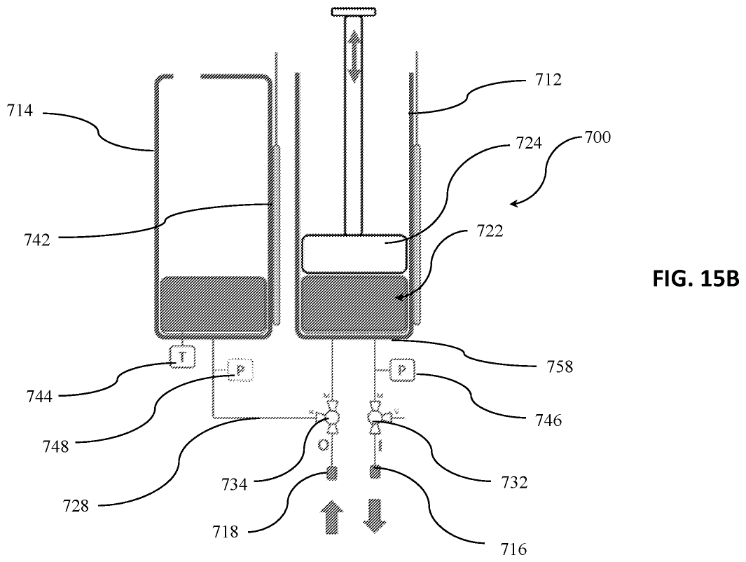

FIG. 15A illustrates a non-limiting embodiment of yet another infusion device 700 such as, for example, infusion device 708 for regulating fluid flow in an expandable member such as, for example, expandable member 707. In a non-limiting exemplary embodiment, the infusion device 700 may include several components that are substantially the same as or similar to the components of other exemplary infusion devices described herein. All such same or similar components are designated by like numerals. In the interest of brevity, the following description of infusion device 700 focuses primarily on those components that are substantially different and/or operate or function differently from those of other infusion devices described herein.

In a non-limiting embodiment, a system 701 for regulating fluid flow in a system used for tissue ablation. In some embodiments, the system 701 may include an elongated body 702 having one or more inflow lumens 703 and one or more outflow lumens 704 extending between a first end 705 and a second end 706 of the body 702. In certain embodiments, the system 701 may include an inflatable member 707 in fluid communication with the inflow and outflow lumens 703 and 704, respectively, proximate the first end 705 of the body 702. In some embodiments, the inflatable member 707 can be non-compliant, i.e., made of a material that resists stretching. When inflating and ablating tissue using inflatable member 707, which can be non-compliant, the flow of fluid therethrough can be effectively controlled by controlling the flow rate of fluid exiting the infusion device 708 as the inflatable member 707 does not apply restorative pressures on the fluid flowing through.

FIG. 15B illustrates a non-limiting embodiment of an infusion device 700 generically illustrated as infusion device 701 in FIG. 15A, for regulating fluid flow in an inflatable member such as, for example, inflatable member 707 of infusion system 701 in FIG. 15A. In a non-limiting exemplary embodiment, infusion device 700 can include a chamber 712 and a heating chamber 714. The infusion device 710 may be coupled to a second end (e.g., second end 706 in FIG. 15A) of an elongated body (e.g., elongated body 702 in FIG. 15A) having inflow and outflow lumens 716 and 718 (e.g., inflow and outflow lumens 703 and 704 in FIG. 15A). The infusion device 700 can include a piston or plunger 724 operatively coupled to a prime mover (not shown), e.g., a stepper motor, which can be slidably disposed within the chamber 712 for regulating the pressure therewithin. In some embodiments, pressurizing the chamber 712 can direct the fluid 722 from the chamber 712 into the inflatable member (not shown) via the inflow lumen 716.

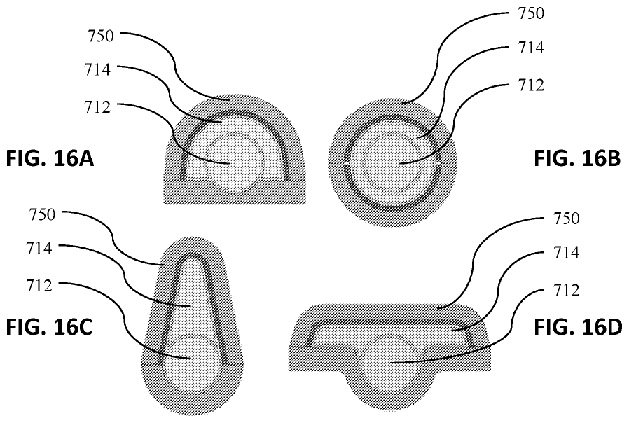

In a non-limiting embodiment, the heating chamber 714 may be vented to allow a volume of fluid heated therein to flow into the chamber 712 and to the inflatable member via the inflow lumen. In a non-limiting embodiment, the heating chamber 714 can include one or more energy sources 742 and may also include one or more temperature sensors 744 disposed within the fluid and operatively coupled to a controller (not shown). In some embodiments, the controller may operate the one or more energy sources 742, e.g., a thermal heat source, to heat and/or cool the fluid within the heating chamber 714 in response to the sensed temperatures from the one or more temperature sensors 744. In some embodiments, the infusion device 700 can have a number of configurations. For example, as seen in FIG. 16A, the heating chamber 714 can partially, or fully, surround the chamber 712 and the entire system can include insulation 750 surrounding both chambers 712, 714. As illustrated, the heating chamber 714 can have a variety of cross-sectional shapes, while the chamber 712 can have a generally circular cross-sectional shape. For example, in FIG. 16A, the heating chamber 714 can have a generally semi-circular arc shape; in FIG. 16B, the heating chamber 714 can have a generally tubular shape; in FIG. 16C, the heating chamber can have a generally triangular shape, with one side having an arc shaped to fit the chamber 712; in FIG. 16D, the heating chamber 714 can have a generally rectangular shape with an arc shaped to fit the chamber 712. Other shapes of heating chambers 714 are considered to be within the scope of this disclosure.

In a non-limiting embodiment, the infusion device 700 can generally include a transfer lumen 728 having a three-way shut-off valve 732 in the inflow lumen 716 and a three-way shut-off valve 734 connecting the chamber 712, the heating chamber 714, and the outflow lumen 718.

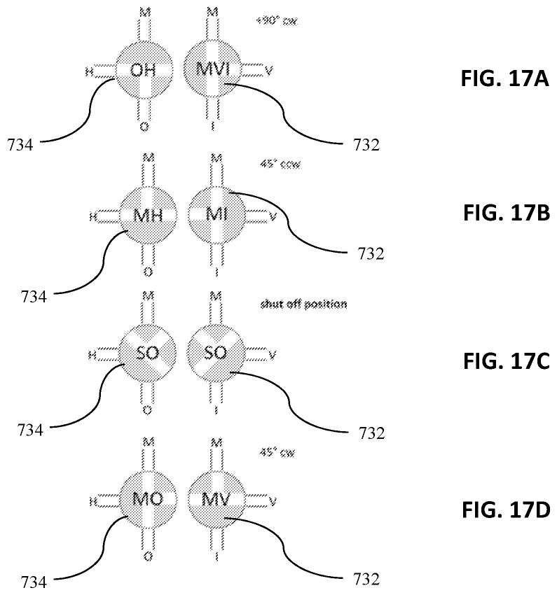

The inflatable member, for example inflatable member 707 as shown in FIG. 15A, can be expanded and/or collapsed by appropriately adjusting the pressure within the chamber 712 and the orientation of the three-way shut-off valves 732, 734. For expanding or inflating the inflatable member, for example during treatment, chamber 712 can be filled with a fluid and the valve 732 may be opened between to establish fluid communication between the chamber 712 and the inflow lumen 716 for directing pressurized fluid into the inflatable member, e.g., as a pre-inflation step, as shown in FIG. 17A. The shut-off valves 732, 734 can be closed and fluid within the heating chamber 714 can be heated to a desired temperature. The shut off-valve 734, as seen in FIG. 17B, can be opened between the heating chamber 714, designated as H, and the chamber 712, designated as M, to allow the piston 724 to draw heated fluid into the chamber 712. The piston 724 can be advanced towards a distal end 758 of the chamber 712 to push the heated fluid through the shut-off valve 732, as seen in FIG. 17B, and the inflow lumen 716, designated as I, towards the inflatable member, for ablation. The fluid can return from the inflatable member through the outflow lumen 716 towards the shut-off valve 734 to be directed back towards the heating chamber 714. During this process, the fluid can be pushed by the piston 724 through the system. When the system times out, i.e., the chamber 712 is emptied of fluid 722, the shut-off valves can be turned to a shut off position, as seen in FIG. 17C, then to a deflation position as seen in FIG. 17D. In the deflation position, the shut-off valves can be open between the inflatable member and the chamber 712 and allowed to vent the remaining fluid, e.g., gas, in the inflatable member to deflate the inflatable member, as the piston 724 is drawn away from the distal end 758 of the chamber 712. If needed, this process can be repeated until the desired amount of ablation is completed.

In a non-limiting embodiment, one or more pressure sensors 746, 748 may be operatively coupled to a controller (not shown). In some embodiments, the controller may be the same as the controller for heating and/or cooling the fluid 722 within the heating chamber 714. In certain embodiments, the one or more pressure sensors 746, 748 may be coupled to a separate controller. In some embodiments, the one or more pressure sensors 746, 748 may be used for monitoring and/or displaying the pressures. In certain embodiments, the one or more pressure sensors 746, 748 may be used for monitoring and/or displaying the pressures and for operating the infusion device 700 by controlling the operational status of one or more components such as, for instance, one or more valves, one or more prime movers, etc.

Although the present disclosure and its advantages have been described in detail, it should be understood that various changes, substitutions, and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A system for regulating fluid flow, the system comprising:
an elongated body having an inflow lumen and an outflow lumen extending between a first end and a second end of the body;
an expandable member in fluid communication with the inflow lumen and the outflow lumen at the first end of the body; and
an infusion device coupled to the second end of the body, the infusion device having (i) a first chamber configured to direct fluid flow, at a first pressure, from within the first chamber into the expandable member via the inflow lumen; and (ii) a second chamber configured to regulate the fluid flow by applying a second pressure on the fluid flow through the outflow lumen.

2. The system of claim 1, wherein
the expandable member inflates when fluid flow into the expandable member is greater than fluid flow out of the expandable member; and
the expandable member deflates when fluid flow into the expandable member is less than fluid flow out of the expandable member.

3. (canceled)

4. The system of claim 1, further comprising a piston slidably disposed within the second chamber, wherein
the piston divides the second chamber into a first section and a second section, the first section being in fluid communication with the outflow lumen,
displacement of the piston towards the first section decreases a volume of the first section and increases the second pressure; and
displacement of the piston towards the second section increases a volume of the first section and decreases the second pressure.

5. (canceled)

6. (canceled)

7. The system of claim 4, further comprising a back pressure chamber having
a first section and a second section, the second section configured for being pressurized and de-pressurized;
operatively coupled first and second pistons, the first piston slidably disposed within the first section and the second piston slidably disposed within the second section; and
a biasing element operatively coupled to the first and second pistons for displacing the first and second pistons to increase or decrease a pressure in the first section of the back pressure chamber.

8. The system of claim 7, further comprising one or more orifices disposed in the outflow lumen for regulating fluid flow into the second chamber.

9. The system of claim 1, wherein the second chamber further comprises
a first section and a second section, the second section configured for being pressurized and de-pressurized;
operatively coupled first and second pistons, the first piston slidably disposed within the first section and the second piston slidably disposed within the second section; and
a prime mover operatively coupled to the first and second pistons for displacing the first and second pistons to increase or decrease the second pressure in the first section of the second chamber.

10. The system of claim 1, wherein the second chamber further comprises
a first section and a second section, the second section configured for being pressurized and de-pressurized;
operatively coupled first and second pistons, the first piston slidably disposed within the first section and the second piston slidably disposed within the second section; and
a biasing element operatively coupled to the first and second pistons for displacing the first and second pistons to increase or decrease the second pressure in the first section of the second chamber.

11. The system of claim 10, further comprising one or more orifices disposed in the outflow lumen for regulating fluid flow into the second chamber.

12. The system of claim 1, wherein the second chamber further comprises
a first enclosure and a second enclosure; and
operatively coupled first and second pistons, wherein
the first piston is slidably disposed within the first enclosure and divides the first enclosure into a first section and a second section;
the second piston is slidably disposed within the second enclosure and divides the second enclosure into a third section and a fourth section;
pressurizing the first section and de-pressurizing the third section increases the second pressure within the first section of the second chamber; and
de-pressurizing the first section and pressurizing the third section decreases the second pressure within the first section of the second chamber.

13. The system of claim 1, further comprising
a piston slidably disposed between the first and second chambers; and
a prime mover operatively coupled to the piston for displacing the piston to increase or decrease the first and second pressures.

14. (canceled)

15. (canceled)

16. The system of claim 1, further comprising
a third chamber configured for pressurization and de-pressurization;
a fourth chamber configured for pressurization and de-pressurization;
a first piston slidably disposed between the first and second chambers; and
a second piston slidably disposed between the third and fourth chambers;
wherein, the first and second pistons are operatively coupled such that displacing the first and second pistons increases or decreases the first and second pressures.

17. The system of claim 16, further comprising a pressurization source in fluid communication with the third and fourth chambers, the system configured for concurrently pressurizing one of the third and fourth chambers and de-pressurizing the other of the third and fourth chambers.

18. (canceled)

19. The system of claim 1, further comprising
at least one controller;
at least one heating element disposed within the fluid in the first chamber; and
at least one temperature sensor disposed within the fluid in the first chamber;
wherein the at least one heating element and the at least one temperature sensor are operatively coupled to the at least one controller configured for maintaining a temperature of the fluid in the first chamber within a predefined range.

20. The system of claim 1, further comprising a pre-inflation chamber configured to direct fluid, in the presence of a third pressure, into and out of the expandable member via a pre-inflation lumen in fluid communication with the expandable member via the outflow lumen.

21. The system of claim 20, further comprising:
a first piston slidably disposed within the first chamber, the first piston dividing the first chamber into a first section and a second section, the first section being in fluid communication with the inflow lumen, and wherein
displacement of the first piston towards the first section of the first chamber decreases a volume of the first section of the first chamber and thereby increases the first pressure; and
displacement of the piston towards the second section of the first chamber increases a volume of the first section of the first chamber and thereby decreases the first pressure;
a second piston slidably disposed within the second chamber, the second piston dividing the second chamber into a first section and a second section, the first section being in fluid communication with the outflow lumen, and wherein
displacement of the second piston towards the first section of the second chamber decreases a volume of the first section of the second chamber and thereby increases the second pressure; and
displacement of the second piston towards the second section of the second chamber increases a volume of the first section of the second chamber and thereby decreases the second pressure; and
a third piston slidably disposed within the pre-inflation chamber, the third piston dividing the pre-inflation chamber into a first section and a second section, the first section being in fluid communication with the pre-inflation lumen, and wherein
displacement of the third piston towards the first section of the pre-inflation chamber decreases a volume of the first section of the pre-inflation chamber and thereby increases the third pressure; and
displacement of the third piston towards the second section of the pre-inflation chamber increases a volume of the first section of the pre-inflation chamber and thereby decreases the third pressure.

22.-23. (canceled)

24. The system of claim 21, wherein, during a procedure, the expandable member is inflated or deflated by regulating the flow of fluid through the expandable member and by inhibiting the flow of fluid into the pre-inflation chamber.

25. The system of claim 24, wherein the expandable member is configured to inflate when fluid flow into the expandable member is greater than fluid flow out of the expandable member.

26. The system of claim 25, wherein fluid from the expandable member is configured to be extracted into the first and/or second chambers and thereby deflate the expandable member.

27. The system of claim 26, further comprising a transfer lumen extending between the first and second chambers configured for transferring fluid from the second chamber to the first chamber after deflating the expandable member at the end of the medical procedure.

28. (canceled)

29. The system of claim 20, further comprising
at least one controller;
at least one heating element disposed within the fluid in the first chamber; and
at least one temperature sensor disposed within the fluid in the first chamber;
wherein the at least one heating element and the at least one temperature sensor are operatively coupled to the at least one controller configured for maintaining a temperature of the fluid in the first chamber within a predefined range.

30-61. (canceled)
